(19) United States
(12) Patent Application Publication
(10) Pub. No.: US 2025/0352596 A1
(43) Pub. Date: Nov. 20, 2025

(54) DIETARY SUPPLEMENTS FOR MOOD SUPPORT

(71) Applicant: Melaleuca, Inc., Idaho Falls, ID (US)

(72) Inventors: Jordan Joy, Idaho Falls, ID (US); Clinton Dahlberg, Idaho Falls, ID (US)

(21) Appl. No.: 19/207,918

(22) Filed: May 14, 2025

Related U.S. Application Data
(60) Provisional application No. 63/647,946, filed on May 15, 2024.
(60) Provisional application No. 63/648,071, filed on May 15, 2024.

(51) Int. Cl. A61K 36/53; A61K 31/047; A61K 31/197; A61K 31/4045; A61K 31/519; A61K 31/714; A61K 36/28; A61K 36/81; A61K 36/88

(52) U.S. Cl. CPC A61K 36/53; A61K 31/047; A61K 31/197; A61K 31/4045; A61K 31/519; A61K 31/714; A61K 36/28; A61K 36/81; A61K 36/88

(57) ABSTRACT

Mood support compositions are described herein. For example, this document provides mood support compositions containing lavender, lemon balm, and chamomile. Such compositions may be used to, e.g., support a positive mental outlook, promote emotional health, support general well-being, and reduce feelings of occasional stress.

### CROSS-REFERENCE TO RELATED APPLICATIONS

This application claims the benefit of U.S. Provisional Application No. 63/647,946, filed on May 15, 2024, and U.S. Provisional Application No. 63/648,071, filed on May 15, 2024, the disclosures of which are incorporated herein by reference in their entirety.

### TECHNICAL FIELD

This document relates to the field of mood support compositions. For example, this document relates to mood support compositions useful for one or more of supporting a positive mental outlook, promoting emotional health, supporting general well-being, and reducing feelings of occasional stress.

### BACKGROUND

Mental health issues are a very real brain health concern. Neurotransmitters such as serotonin can affect mood and one's ability to cope with stress. Proper vitamins and supplementation can support the brain as it functions throughout life and help the produce the proper amounts of necessary chemicals like serotonin. If the brain is receiving proper nutrients, it can start to send signals that something is wrong. For example, some of the signals the brain can send are anxiousness, feelings of being overwhelmed, or unshakable feeling of sadness. There is a continuing need to formulate compositions that support and/or improve mood.

### SUMMARY

Provided herein are mood support compositions comprising: a lavender extract; a lemon balm extract; a chamomile extract; and inositol.

In some embodiments, the lavender extract is present in an amount of about 12 mg to about 1220 mg per serving of the mood support extract. In some embodiments, the lavender extract is present in an amount of about 49 mg to about 490 mg per serving of the mood support extract.

In some embodiments, the lemon balm extract is present in an amount of about 2 mg to about 293 mg per serving of the mood support extract. In some embodiments, the lemon balm extract is present in an amount of about 11 mg to about 117 mg per serving of the mood support extract.

In some embodiments, the chamomile extract is present in an amount of about 2 mg to about 244 mg per serving of the mood support extract. In some embodiments, the chamomile extract is present in an amount of about 9 mg to about 98 mg per serving of the mood support extract.

In some embodiments, the inositol is present in an amount of about 2 mg to about 244 mg per serving of the mood support extract. In some embodiments, the inositol is present in an amount of about 9 mg to about 98 mg per serving of the mood support extract.

In some embodiments, the mood support composition further comprises Composition B (e.g., any Composition B described herein). For example, in some embodiments, the mood support composition further comprises an ashwagandha extract; a folate; and vitamin B12, wherein the vitamin B12 comprises methylcobalamin. In some embodiments, the mood support composition further comprises a saffron extract; an ashwagandha extract; a folate; and vitamin B12, wherein the vitamin B12 comprises methylcobalamin.

In some embodiments, the saffron extract is present in an amount of about 14 mg to about 56 mg per serving of the mood support composition. In some embodiments, the saffron extract is present in an amount of about 28 mg to about 30 mg per serving of the mood support composition. In some embodiments, the saffron extract is present in an amount of about 28 mg per serving of the mood support composition.

In some embodiments, the saffron extract is an extract of the stigmas of Crocus sativus flowers.

In some embodiments, the mood support composition does not comprise a saffron extract.

In some embodiments, the ashwagandha extract is present in an amount of about 125 mg to about 1800 mg per serving of the mood support composition. In some embodiments, the ashwagandha extract is present in an amount of about 300 mg to about 675 mg per serving of the mood support composition. In some embodiments, the ashwagandha extract is present in an amount of about 600 mg per serving of the mood support composition.

In some embodiments, the ashwagandha extract contains one or more withanolides.

In some embodiments, the ashwagandha extract is an ashwagandha root extract.

In some embodiments, vitamin B12 is present in an amount of about 0.5 μg to about 100 μg per serving of the mood support composition. In some embodiments, vitamin B12 is present in an amount of about 3 μg to about 60 μg per serving of the mood support composition. In some embodiments, vitamin B12 is present in an amount of about 6 μg per serving of the mood support composition.

In some embodiments, the folate is present in an amount of about 0.1 mg to about 2 mg DFE per serving of the mood support composition. In some embodiments, the folate is present in an amount of about 0.2 mg to about 0.8 mg DFE per serving of the mood support composition. In some embodiments, the folate is present in an amount of about 0.4 mg DFE per serving of the mood support composition.

In some embodiments, the folate comprises calcium methyltetrahydrofolate.

In some embodiments, the mood support composition further comprises Composition C (e.g., any Composition C described herein). For example, in some embodiments, the mood support composition further comprises: melatonin; L-theanine; GABA (gamma-aminobutyric acid); and an ashwagandha extract.

In some embodiments, the melatonin is present in an amount of about 1 mg to about 10 mg per serving. In some embodiments, the melatonin is present in an amount of about 3 mg to about 7 mg per serving. In some embodiments, the melatonin is present in an amount of about 5 mg per serving.

In some embodiments, the mood support composition is formulated for rapid release and sustained release of the melatonin. In some embodiments, about 20% to about 60% w/w of the melatonin is released within a first period of time. In some embodiments, about 40% w/w of the melatonin is released within a first period of time.

In some embodiments, the first period of time is about 30 minutes to about 1 hour. In some embodiments, the first period of time is about 1 hour.

In some embodiments, about 40% to about 80% w/w of the melatonin is released over a second period of time following the first period of time. In some embodiments, about 60% w/w of the melatonin is released within the second period of time.

In some embodiments, the second period of time is about 6 hours to about 8 hours. In some embodiments, the second period of time is about 7 hours.

In some embodiments, the L-theanine is present in an amount of about 25 mg to about 500 mg per serving. In some embodiments, the L-theanine is present in an amount of about 100 mg to about 300 mg per serving. In some embodiments, the L-theanine is present in an amount of about 200 mg per serving.

In some embodiments, the GABA is present in an amount of about 100 mg to about 1000 mg per serving. In some embodiments, the GABA is present in an amount of about 200 mg to about 600 mg per serving. In some embodiments, the GABA is present in an amount of about 300 mg per serving.

In some embodiments, the ashwagandha extract is present in an amount of about 50 mg to about 1800 mg per serving. In some embodiments, the ashwagandha extract is present in an amount of about 125 mg to about 675 mg per serving. In some embodiments, the ashwagandha extract is present in an amount of about 125 mg per serving.

In some embodiments, the ashwagandha extract contains one or more withanolides. In some embodiments, the one or more withanolides are present in an amount of about 1% to about 12% w/w of the ashwagandha extract. In some embodiments, the one or more withanolides are present in an amount of about 5% w/w of the ashwagandha extract.

In some embodiments, the mood support composition further comprises a valerian root extract.

In some embodiments, the lavender and lemon balm are present at a ratio of about 25:1 to about 1:1 lavender:lemon balm. In some embodiments, the lavender and lemon balm are present at a ratio of about 5:1 to about 1:1 lavender:lemon balm. In some embodiments, the lavender and lemon balm are present at a ratio of about 4:1 lavender:lemon balm.

In some embodiments, the chamomile and inositol are present at a ratio of about 25:1 to about 1:25 chamomile:inositol. In some embodiments, the chamomile and inositol are present at a ratio of about 2:1 to about 1:2 chamomile:inositol. In some embodiments, the chamomile and inositol are present at a ratio of about 1:1 chamomile:inositol.

In some embodiments, the lavender and lemon balm and the chamomile and inositol are present at a ratio of about 25:1 to about 1:1 lavender and lemon balm: chamomile and inositol. In some embodiments, the lavender and lemon balm and the chamomile and inositol are present at a ratio of about 4:1 to about 1:1 lavender and lemon balm: chamomile and inositol.

In some embodiments, the lavender and lemon balm and the chamomile and inositol are present at a ratio of about 3:1 lavender and lemon balm: chamomile and inositol.

In some embodiments, the mood support composition is formulated as a tablet, a capsule, or a soft gel. In some embodiments, two tablets, capsules, or soft gels comprises a full serving of the mood support composition. In some embodiments, the mood support composition is formulated as a capsule.

Also provided herein are methods of improving one or more aspects related to mood in a mammal, the method comprising administering to the mammal any of the mood support compositions provided herein.

In some embodiments, improving an aspect related to mood comprises one or more of improving emotional health, improving the ability to cope with stress, improving the activity of a brain messenger, improving emotional balance, improving general well-being, improving the restfulness of sleep, and improving physical and mental energy.

Also provided herein are methods of reducing feelings of sadness and/or feelings of stress in a mammal, the method comprising administering to the mammal any of the mood support compositions provided herein.

Also provided herein are methods of inhibiting GSK3α and/or GSK3β in a mammal, the method comprising administering to the mammal any of the mood support compositions provided herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a plot showing an exemplary GSK3β response curve with IC50 data for Blend A.

FIG. 2 is a plot showing an exemplary GSK3ß response curve with IC50 data for staurosporine.

FIG. 3 is a plot showing an exemplary GSK3α response curve with IC50 data for lavender, chamomile, lemon balm, Blend A, and inositol.

FIG. 4 is a plot showing an exemplary GSK3α response curve with IC50 data for staurosporine.

FIG. 5 is a plot showing GSK3ß inhibitory contribution relative to expected for Blend A (“Blend”) and each of lavender, chamomile, lemon balm, and inositol.

### DETAILED DESCRIPTION

This document provides mood support compositions that contain lavender (e.g., a lavender extract), lemon balm (e.g., a lemon balm extract), and chamomile (e.g., a chamomile extract). Also provided herein are mood support compositions containing lavender (e.g., a lavender extract), lemon balm (e.g., a lemon balm extract), chamomile (e.g., a chamomile extract), and inositol. In some embodiments, a mood support composition provided herein that contains lavender (e.g., a lavender extract), lemon balm (e.g., a lemon balm extract), chamomile (e.g., a chamomile extract), and optionally inositol (e.g., Composition A) can be combined with one or more other compositions (e.g., one or more other mood support compositions and/or sleep aid compositions). For example, a mood support composition provided herein can comprise Composition A and Composition B and/or Composition C as described herein.

The mood support compositions provided herein can be useful for one or more of supporting a positive mental outlook, better coping with the ups and downs of life, maintaining a balanced mood, promoting emotional health, supporting emotional balance, supporting the activities of brain messengers, supporting general well-being, supporting more restful sleep (e.g., restful sleep without sedation), reducing feelings of increased sadness, reducing feelings of occasional stress, supporting mood-related sexual health, and promoting physical and mental energy (e.g., after consuming a mood support composition provided herein as compared to not taking a mood support composition provided herein).

### Composition A

### Lavender

The mood support compositions provided herein include lavender. Lavender is an herb that is commonly used in calming blends as an inhalant or taken internally in teas and ingestible tablets. Lavender can improve mood, lower heart rate, lower adrenaline levels, regulate breathing, and improve sleep quality. Lavender is suggested to modulate GABAergic (gamma-aminobutyric acid or γ-aminobutyric acid) neurotransmission, particularly on GABA_{A} receptors, and enhance an inhibitory tone of the nervous system. The interaction of lavender on the cholinergic system, one of the most important neurotransmitter systems in the brain, is postulated to play a role in its antianxiety and antidepression effects. Lavender can interact with glutaminergic and NMDA receptors, interact with the serotonin receptor, and/or have SSRI-like activity by reducing expression of 5HT-1A receptors within the brain.

Lavender, as used herein, can include dried flowers and/or an extract from one or more members of the Lavandula genus. Non-limiting examples of Lavandula genus members include L. stoechas, L. pedunculata, L. dentata, L. multifida., L. latifolia, and L. angustifolia. A lavender extract can be in the form of a liquid (e.g., an aqueous solution), a solid (e.g., a particulate or a powder), or a semi-solid form (e.g., a gel or an emulsion). Any appropriate method can be used to obtain a lavender extract for use in the mood support compositions provided herein. For example, a lavender extract can be obtained by aqueous or ethanolic extraction from flowers from one or more members of the Lavandula genus. In some embodiments, a lavender extract is an extract prepared from the flowers of L. angustifolia.

The mood support compositions provided herein can contain any appropriate amount of a lavender extract. In some embodiments, a lavender extract is present in an amount of about 12 mg to about 1220 mg per serving of a mood support composition provided herein (e.g., per serving of Composition A). For example, a lavender extract can be present in an amount of about 12 mg to about 100 mg, about 12 mg to about 200 mg, about 12 mg to about 300 mg, about 12 mg to about 400 mg, about 12 mg to about 500 mg, about 12 mg to about 600 mg, about 12 mg to about 700 mg, about 12 mg to about 800 mg, about 12 mg to about 900 mg, about 12 mg to about 1000 mg, about 12 mg to about 1100 mg, about 100 mg to about 200 mg, about 100 mg to about 300 mg, about 100 mg to about 400 mg, about 100 mg to about 500 mg, about 100 mg to about 600 mg, about 100 mg to about 700 mg, about 100 mg to about 800 mg, about 100 mg to about 900 mg, about 100 mg to about 1000 mg, about 100 mg to about 1100 mg, about 100 mg to about 1220 mg, about 200 mg to about 300 mg, about 200 mg to about 400 mg, about 200 mg to about 500 mg, about 200 mg to about 600 mg, about 200 mg to about 700 mg, about 200 mg to about 800 mg, about 200 mg to about 900 mg, about 200 mg to about 1000 mg, about 200 mg to about 1100 mg, about 200 mg to about 1220 mg, about 300 mg to about 400 mg, about 300 mg to about 500 mg, about 300 mg to about 600 mg, about 300 mg to about 700 mg, about 300 mg to about 800 mg, about 300 mg to about 900 mg, about 300 mg to about 1000 mg, about 300 mg to about 1100 mg, about 300 mg to about 1220 mg, about 400 mg to about 500 mg, about 400 mg to about 600 mg, about 400 mg to about 700 mg, about 400 mg to about 800 mg, about 400 mg to about 900 mg, about 400 mg to about 1000 mg, about 400 mg to about 1100 mg, about 400 mg to about 1220 mg, about 500 mg to about 600 mg, about 500 mg to about 700 mg, about 500 mg to about 800 mg, about 500 mg to about 900 mg, about 500 mg to about 1000 mg, about 500 mg to about 1100 mg, about 500 mg to about 1220 mg, about 600 mg to about 700 mg, about 600 mg to about 800 mg, about 600 mg to about 900 mg, about 600 mg to about 1000 mg, about 600 mg to about 1100 mg, about 600 mg to about 1220 mg, about 700 mg to about 800 mg, about 700 mg to about 900 mg, about 700 mg to about 1000 mg, about 700 mg to about 1100 mg, about 700 mg to about 1220 mg, about 800 mg to about 900 mg, about 800 mg to about 1000 mg, about 800 mg to about 1100 mg, about 800 mg to about 1220 mg, about 900 mg to about 1000 mg, about 900 mg to about 1100 mg, about 900 mg to about 1220 mg, about 1000 mg to about 1100 mg, about 1000 mg to about 1220 mg or about 1100 mg to about 1220 mg per serving of a mood support composition provided herein. In some embodiments, a lavender extract is present in an amount of about 49 mg to about 490 mg per serving of a mood support composition provided herein. In some embodiments, a lavender extract is present in an amount of about 75 mg to about 175 mg per serving of a mood support composition provided herein. In some embodiments, a lavender extract is present in an amount of about 100 mg to about 150 mg per serving of a mood support composition provided herein.

In some embodiments, a lavender extract is present in an amount of about 12 mg, about 50 mg, about 100 mg, about 125 mg, about 150 mg, about 175 mg, about 200 mg, about 300 mg, about 400 mg, about 500 mg, about 600 mg, about 700 mg, about 800 mg, about 900 mg, about 1000 mg, about 1100 mg, or about 1220 mg per serving of a mood support composition provided herein.

In some embodiments, a lavender extract is present in an amount of about 25% to about 98% w/w of Composition A. For example, a lavender extract can be present in an amount of about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 25% to about 80%, about 25% to about 85%, about 25% to about 90%, about 25% to about 95%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 30% to about 85%, about 30% to about 90%, about 30% to about 95%, about 30% to about 98%, about 35% to about 40%, about 35% to about 45%, about 35% to about 50%, about 35% to about 55%, about 35% to about 60%, about 35% to about 65%, about 35% to about 70%, about 35% to about 75%, about 35% to about 80%, about 35% to about 85%, about 35% to about 90%, about 35% to about 95%, about 35% to about 98%, about 40% to about 45%, about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 95%, about 40% to about 98%, about 45% to about 50%, about 45% to about 55%, about 45% to about 60%, about 45% to about 65%, about 45% to about 70%, about 45% to about 75%, about 45% to about 80%, about 45% to about 85%, about 45% to about 90%, about 45% to about 95%, about 45% to about 98%, about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to about 98%, about 55% to about 60%, about 55% to about 65%, about 55% to about 70%, about 55% to about 75%, about 55% to about 80%, about 55% to about 85%, about 45% to about 90%, about 45% to about 95%, about 45% to about 98%, about 60% to about 65%, about 60% to about 70%, about 60% to about 75%, about 60% to about 80%, about 60% to about 85%, about 60% to about 90%, about 60% to about 95%, about 60% to about 98%, about 65% to about 70%, about 65% to about 75%, about 65% to about 80%, about 65% to about 85%, about 65% to about 90%, about 65% to about 95%, about 65% to about 98%, about 70% to about 75%, about 70% to about 80%, about 70% to about 85%, about 70% to about 90%, about 70% to about 95%, about 70% to about 98%, about 75% to about 80%, about 75% to about 85%, about 75% to about 90%, about 75% to about 95%, about 75% to about 98%, about 80% to about 85%, about 80% to about 90%, about 80% to about 95%, about 80% to about 98%, about 85% to about 90%, about 85% to about 95%, about 85% to about 98%, about 90% to about 95%, about 90% to about 98%, or about 95% to about 98% w/w of Composition A. In some embodiments, a lavender extract is present in an amount of about 50% to about 80% or about 55% to about 65% w/w of Composition A.

In some embodiments, a lavender extract is present in an amount of about 25%, about 30%, about 35%, about 40%, about 45%, about 50%, about 55%, about 56%, about 57%, about 58%, about 59%, about 60%, about 61%, about 62%, about 63%, about 64%, about 65%, about 70%, about 75%, about 80%, about 85%, about 90%, about 95% or about 98% w/w of Composition A. In some embodiments, a lavender extract is present in an amount of about 61% w/w of Composition A.

### Lemon Balm

The mood support compositions provided herein include lemon balm. Lemon Balm (Melissa officinalis) is a perennial herbaceous plant in the mint family. It is native to south-central Europe, the Mediterranean Basin, Iran, and Central Asia. Lemon balm can have calming and mood-enhancing properties. It has been used for centuries to promote relaxation, reduce stress, and improve sleep. Lemon balm is believed to work by increasing the levels of GABA in the brain, which can help reduce anxiety and induce a sense of calmness. It may also have mild sedative effects, making it beneficial for promoting better sleep quality.

Lemon balm, as used in a mood support composition provided herein, includes dried lemon balm leaves, dried lemon balm stems, and/or a lemon balm extract. Any appropriate method can be used to obtain a lemon balm extract for use in the mood support compositions provided herein. For example, a lemon balm extract can be obtained by aqueous or ethanolic extraction from lemon balm leaves and/or stems.

The mood support compositions provided herein can contain any appropriate amount of lemon balm. In some embodiments, a lemon balm extract is present in an amount of about 2 mg to about 293 mg per serving of a mood support composition provided herein (e.g., per serving of Composition A). For example, a lemon balm extract can be present in an amount of about 2 mg to about 10 mg, about 2 mg to about 30 mg, about 2 mg to about 50 mg, about 2 mg to about 75 mg, about 2 mg to about 100 mg, about 2 mg to about 125 mg, about 2 mg to about 150 mg, about 2 mg to about 175 mg, about 2 mg to about 200 mg, about 2 mg to about 225 mg, about 2 mg to about 250 mg, about 2 mg to about 275 mg, about 10 mg to about 30 mg, about 10 mg to about 40 mg, about 10 mg to about 60 mg, about 10 mg to about 80 mg, about 10 mg to about 100 mg, about 10 mg to about 125 mg, about 10 mg to about 150 mg, about 10 mg to about 175 mg, about 10 mg to about 200 mg, about 10 mg to about 225 mg, about 10 mg to about 250 mg, about 10 mg to about 275 mg, about 10 mg to about 293 mg, about 30 mg to about 50 mg, about 30 mg to about 75 mg, about 30 mg to about 100 mg, about 30 mg to about 125 mg, about 30 mg to about 150 mg, about 30 mg to about 175 mg, about 30 mg to about 200 mg, about 30 mg to about 225 mg, about 30 mg to about 250 mg, about 30 mg to about 275 mg, about 30 mg to about 293 mg, about 50 mg to about 75 mg, about 50 mg to about 100 mg, about 50 mg to about 125 mg, about 50 mg to about 150 mg, about 50 mg to about 175 mg, about 50 mg to about 200 mg, about 50 mg to about 225 mg, about 50 mg to about 250 mg, about 50 mg to about 275 mg, about 50 mg to about 293 mg, about 75 mg to about 100 mg, about 75 mg to about 125 mg, about 75 mg to about 150 mg, about 75 mg to about 175 mg, about 75 mg to about 200 mg, about 75 mg to about 225 mg, about 75 mg to about 250 mg, about 75 mg to about 275 mg, about 75 mg to about 293 mg, about 75 mg to about 125 mg, about 75 mg to about 150 mg, about 100 mg to about 175 mg, about 100 mg to about 200 mg, about 100 mg to about 225 mg, about 100 mg to about 250 mg, about 100 mg to about 275 mg, about 100 mg to about 293 mg, about 125 mg to about 150 mg, about 125 mg to about 175 mg, about 125 mg to about 200 mg, about 125 mg to about 225 mg, about 125 mg to about 250 mg, about 125 mg to about 275 mg, about 125 mg to about 293 mg, about 150 mg to about 175 mg, about 150 mg to about 200 mg, about 150 mg to about 225 mg, about 150 mg to about 250 mg, about 150 mg to about 275 mg, about 150 mg to about 293 mg, about 175 mg to about 200 mg, about 175 mg to about 225 mg, about 175 mg to about 250 mg, about 175 mg to about 275 mg, about 175 mg to about 293 mg, about 200 mg to about 225 mg, about 200 mg to about 250 mg, about 200 mg to about 275 mg, about 200 mg to about 293 mg, about 225 mg to about 250 mg, about 225 mg to about 275 mg, about 225 mg to about 293 mg, about 250 mg to about 275 mg, about 250 mg to about 293 mg, or about 275 mg to about 293 mg per serving of a mood support composition provided herein. In some embodiments, a lemon balm extract is present in an amount of about 11 mg to about 117 mg per serving of a mood support composition provided herein. In some embodiments, a lemon balm extract is present in an amount of about 10 mg to about 40 mg per serving of a mood support composition provided herein. In some embodiments, a lemon balm extract is present in an amount of about 30 mg to about 60 mg per serving of a mood support composition provided herein.

In some embodiments, a lemon balm extract is present in an amount of about 2 mg, about 10 mg, about 30 mg, about 40 mg, about 60 mg, about 80 mg, about 100 mg, about 125 mg, about 150 mg, about 175 mg, about 200 mg, about 225 mg, about 250 mg, about 275 mg, or about 293 mg per serving of a mood support composition provided herein.

In some embodiments, a lemon balm extract is present in an amount of about 1% to about 25% w/w of Composition A. For example, a lemon balm extract can be present in an amount of about 1% to about 5%, about 1% to about 10%, about 1% to about 15%, about 1% to about 20%, about 5% to about 10%, about 5% to about 15%, about 5% to about 20%, about 5% to about 25%, about 10% to about 15%, about 10% to about 20%, about 10% to about 25%, about 15% to about 20%, about 15% to about 25%, or about 20% to about 25% w/w of Composition A. In some embodiments, a lemon balm extract is present in an amount of about 10% to about 20% or about 12% to about 17%/w of Composition A.

In some embodiments, a lemon balm extract is present in an amount of about 1%, about 5%, about 10%, about 12%, about 15%, about 17%, about 20%, about 25% w/w of Composition A. In some embodiments, a lemon balm extract is present in an amount of about 15% w/w of Composition A.

### Chamomile

The mood support compositions provided herein can further include chamomile. Chamomile is an herb that is often consumed as tea to promote relaxation and calmness. Chamomile is commonly used in sleep supplements to help induce sleep, reduce anxiety, and improve sleep quality by promoting a sense of tranquility and calmness. Chamomile can contain compounds that bind to certain receptors in the brain, potentially exerting a mild sedative effect.

Chamomile, as used herein, can include dried flowers and/or an extract from Matricaria chamomilla, Matricaria recutita, and Chamaemelum nobile. Any appropriate method can be used to obtain a chamomile extract for use in the mood support compositions provided herein. For example, a chamomile extract can be obtained by aqueous or ethanolic extraction from chamomile flowers. In some embodiments, a chamomile extract is an extract prepared from the flowers of Matricaria chamomilla.

The mood support compositions provided herein can contain any appropriate amount of chamomile. In some embodiments, a chamomile extract is present in an amount of about 2 mg to about 244 mg per serving of a mood support composition provided herein (e.g., per serving of Composition A). For example, a chamomile extract can be present in an amount of about 2 mg to about 5 mg, 2 mg to about 25 mg, about 2 mg to about 50 mg, about 2 mg to about 75 mg, about 2 mg to about 100 mg, about 2 mg to about 125 mg, about 2 mg to about 150 mg, about 2 mg to about 175 mg, about 2 mg to about 200 mg, about 2 mg to about 225 mg, 5 mg to about 25 mg, about 5 mg to about 50 mg, about 5 mg to about 75 mg, about 5 mg to about 100 mg, about 5 mg to about 125 mg, about 5 mg to about 150 mg, about 5 mg to about 175 mg, about 5 mg to about 200 mg, about 5 mg to about 225 mg, about 5 mg to about 244 mg, about 25 mg to about 50 mg, about 25 mg to about 75 mg, about 25 mg to about 100 mg, about 25 mg to about 125 mg, about 25 mg to about 150 mg, about 25 mg to about 175 mg, about 25 mg to about 200 mg, about 25 mg to about 225 mg, about 25 mg to about 244 mg, about 50 mg to about 75 mg, about 50 mg to about 100 mg, about 50 mg to about 125 mg, about 50 mg to about 150 mg, about 50 mg to about 175 mg, about 50 mg to about 200 mg, about 50 mg to about 225 mg, about 50 mg to about 244 mg, about 75 mg to about 100 mg, about 75 mg to about 125 mg, about 75 mg to about 150 mg, about 75 mg to about 175 mg, about 75 mg to about 200 mg, about 75 mg to about 225 mg, about 75 mg to about 244 mg, about 100 mg to about 125 mg, about 100 mg to about 150 mg, about 100 mg to about 175 mg, about 100 mg to about 200 mg, about 100 mg to about 225 mg, about 100 mg to about 244 mg, about 125 mg to about 150 mg, about 125 mg to about 175 mg, about 125 mg to about 200 mg, about 125 mg to about 225 mg, about 125 mg to about 244 mg, about 150 mg to about 175 mg, about 150 mg to about 200 mg, about 150 mg to about 225 mg, about 150 mg to about 244 mg, about 175 mg to about 200 mg, about 175 mg to about 225 mg, about 175 mg to about 244 mg, about 200 mg to about 225 mg, about 200 mg to about 244 mg, or about 225 mg to about 244 mg per serving of a mood support composition provided herein. In some embodiments, a chamomile extract is present in an amount of about 9 mg to about 98 mg per serving of a mood support composition provided herein. In some embodiments, a chamomile extract is present in an amount of about 10 mg to about 50 mg per serving of a mood support composition provided herein. In some embodiments, a chamomile extract is present in an amount of about 20 mg to about 70 mg per serving of a mood support composition provided herein.

In some embodiments, a chamomile extract is present in an amount of about 2 mg, about 5 mg, about 15 mg, about 20 mg, about 25 mg, about 30 mg, about 40 mg, about 50 mg, about 75 mg, about 100 mg, about 125 mg, about 150 mg, about 175 mg, about 200 mg, about 225 mg, or about 244 mg per serving of a mood support composition provided herein.

In some embodiments, a chamomile extract is present in an amount of about 0.5% to about 25% w/w of Composition A. For example, a chamomile extract can be present in an amount of about 0.5% to about 1%, about 0.5% to about 5%, about 0.5% to about 10%, about 0.5% to about 15%, about 0.5% to about 20%, about 1% to about 5%, about 1% to about 10%, about 1% to about 15%, about 1% to about 20%, about 1% to about 25%, about 5% to about 10%, about 5% to about 15%, about 5% to about 20%, about 5% to about 25%, about 10% to about 15%, about 10% to about 20%, about 10% to about 25%, about 15% to about 20%, about 15% to about 25%, or about 20% to about 25% w/w of Composition A. In some embodiments, a chamomile extract is present in an amount of about 5% to about 15% or about 10% to about 15%/w of Composition A.

In some embodiments, a chamomile extract is present in an amount of about 0.5%, about 1%, about 5%, about 10%, about 12%, about 15%, about 20%, about 25% w/w of Composition A. In some embodiments, a chamomile extract is present in an amount of about 12% w/w of Composition A.

### Inositol

The mood support compositions provided herein can further include inositol. Inositol is a form of sugar that can be naturally produced by the human body to provide structure to cells. Inositol is needed for both proper development and functioning of cells. In addition to its role on the development of cell membranes, inositol has been suggested to have an effect on neurotransmitters in the brain. Individuals having depression or other psychological disorders can have lower levels of brain inositol than matched controls. Increasing inositol consumption via supplementation can improve symptoms of depression, symptoms of pre-menstrual syndrome, and provide mood stability in those with schizophrenia.

Any appropriate method can be used to obtain inositol for use in the mood support compositions provided herein. For example, inositol can be obtained by chemical acid hydrolysis, microbial fermentation, or in vitro enzymatic biocatalysis. In some embodiments, the inositol is myo-inositol.

The mood support compositions provided herein can contain any appropriate amount of inositol. In some embodiments, inositol is present in an amount of about 2 mg to about 244 mg per serving of a mood support composition provided herein (e.g., per serving of Composition A). For example, inositol can be present in an amount of about 2 mg to about 5 mg, 2 mg to about 25 mg, about 2 mg to about 50 mg, about 2 mg to about 75 mg, about 2 mg to about 100 mg, about 2 mg to about 125 mg, about 2 mg to about 150 mg, about 2 mg to about 175 mg, about 2 mg to about 200 mg, about 2 mg to about 225 mg, 5 mg to about 25 mg, about 5 mg to about 50 mg, about 5 mg to about 75 mg, about 5 mg to about 100 mg, about 5 mg to about 125 mg, about 5 mg to about 150 mg, about 5 mg to about 175 mg, about 5 mg to about 200 mg, about 5 mg to about 225 mg, about 5 mg to about 244 mg, about 25 mg to about 50 mg, about 25 mg to about 75 mg, about 25 mg to about 100 mg, about 25 mg to about 125 mg, about 25 mg to about 150 mg, about 25 mg to about 175 mg, about 25 mg to about 200 mg, about 25 mg to about 225 mg, about 25 mg to about 244 mg, about 50 mg to about 75 mg, about 50 mg to about 100 mg, about 50 mg to about 125 mg, about 50 mg to about 150 mg, about 50 mg to about 175 mg, about 50 mg to about 200 mg, about 50 mg to about 225 mg, about 50 mg to about 244 mg, about 75 mg to about 100 mg, about 75 mg to about 125 mg, about 75 mg to about 150 mg, about 75 mg to about 175 mg, about 75 mg to about 200 mg, about 75 mg to about 225 mg, about 75 mg to about 244 mg, about 100 mg to about 125 mg, about 100 mg to about 150 mg, about 100 mg to about 175 mg, about 100 mg to about 200 mg, about 100 mg to about 225 mg, about 100 mg to about 244 mg, about 125 mg to about 150 mg, about 125 mg to about 175 mg, about 125 mg to about 200 mg, about 125 mg to about 225 mg, about 125 mg to about 244 mg, about 150 mg to about 175 mg, about 150 mg to about 200 mg, about 150 mg to about 225 mg, about 150 mg to about 244 mg, about 175 mg to about 200 mg, about 175 mg to about 225 mg, about 175 mg to about 244 mg, about 200 mg to about 225 mg, about 200 mg to about 244 mg, or about 225 mg to about 244 mg per serving of a mood support composition provided herein. In some embodiments, inositol is present in an amount of about 9 mg to about 98 mg per serving of a mood support composition provided herein. In some embodiments, inositol is present in an amount of about 10 mg to about 50 mg per serving of a mood support composition provided herein. In some embodiments, inositol is present in an amount of about 20 mg to about 70 mg per serving of a mood support composition provided herein.

In some embodiments, inositol is present in an amount of about 2 mg, about 5 mg, about 15 mg, about 20 mg, about 25 mg, about 30 mg, about 40 mg, about 50 mg, about 75 mg, about 100 mg, about 125 mg, about 150 mg, about 175 mg, about 200 mg, about 225 mg, or about 244 mg per serving of a mood support composition provided herein.

In some embodiments, inositol is present in an amount of about 0.5% to about 25% w/w of Composition A. For example, inositol can be present in an amount of about 0.5% to about 1%, about 0.5% to about 5%, about 0.5% to about 10%, about 0.5% to about 15%, about 0.5% to about 20%, about 1% to about 5%, about 1% to about 10%, about 1% to about 15%, about 1% to about 20%, about 1% to about 25%, about 5% to about 10%, about 5% to about 15%, about 5% to about 20%, about 5% to about 25%, about 10% to about 15%, about 10% to about 20%, about 10% to about 25%, about 15% to about 20%, about 15% to about 25%, or about 20% to about 25% w/w of Composition A. In some embodiments, inositol is present in an amount of about 5% to about 15% or about 10% to about 15%/w of Composition A.

In some embodiments, inositol is present in an amount of about 0.5%, about 1%, about 5%, about 10%, about 12%, about 15%, about 20%, about 25% w/w of Composition A. In some embodiments, inositol is present in an amount of about 12% w/w of Composition A.

In some embodiments, a mood support composition provided herein (e.g., Composition A) is a composition that includes lavender (e.g., a lavender extract), lemon balm (e.g., a lemon balm extract), chamomile (e.g., a chamomile extract), and inositol.

In some embodiments, lavender and lemon balm are present in a mood support composition provided herein at a ratio of about 25:1 to about 1:1 lavender:lemon balm. For example, lavender and lemon balm can be present in a mood support composition provided herein at a ratio of about 25:1 to about 20:1, about 25:1 to about 15:1, about 25:1 to about 10:1, about 25:1 to about 5:1, about 25:1 to about 2:1, about 20:1 to about 15:1, about 20:1 to about 10:1, about 20:1 to about 5:1, about 20:1 to about 2:1, 20:1 to about 1:1, about 15:1 to about 10:1, about 15:1 to about 5:1, about 15:1 to about 2:1, about 15:1 to about 1:1, about 10:1 to about 5:1, about 10:1 to about 2:1, about 10:1 to about 1:1, about 5:1 to about 2:1, about 5:1 to about 1:1, or about 2:1 to about 1:1 lavender:lemon balm. In some embodiments, lavender and lemon balm are present in a mood support composition provided herein at a ratio of about 5:1 to about 1:1 lavender:lemon balm. In some embodiments, lavender and lemon balm are present in a mood support composition provided herein at a ratio of about 25:1, about 20:1, about 15:1, about 10:1, about 5:1, about 4:1, about 3:1, about 2:1, about 5:3, about 3:2, or about 1:1 lavender:lemon balm. In some embodiments, lavender and lemon balm are present in a mood support composition provided herein at a ratio of about 4:1 lavender:lemon balm.

In some embodiments, chamomile and inositol are present in a mood support composition provided herein at a ratio of about 25:1 to about 1:25 chamomile:inositol. For example, chamomile and inositol can be present in a mood support composition provided herein at a ratio of about 25:1 to about 20:1, about 25:1 to about 15:1, about 25:1 to about 10:1, about 25:1 to about 5:1, about 25:1 to about 2:1, about 20:1 to about 15:1, about 20:1 to about 10:1, about 20:1 to about 5:1, about 20:1 to about 2:1, 20:1 to about 1:1, about 15:1 to about 10:1, about 15:1 to about 5:1, about 15:1 to about 2:1, about 15:1 to about 1:1, about 10:1 to about 5:1, about 10:1 to about 2:1, about 10:1 to about 1:1, about 5:1 to about 2:1, about 5:1 to about 1:1, about 2:1 to about 1:1; about 1:1 to about 1:2, about 1:1 to about 1:5, about 1:1 to about 1:10, about 1:2 to about 1:10, about 1:5 to about 1:10, about 1:1 to about 1:15, about 1:2 to about 1:15, about 1:5 to about 1:15, about 1:10 to about 1:15, about 1:1 to about 1:20, about 1:2 to about 1:20, about 1:5 to about 1:20, about 1:10 to about 1:20, about 1:15 to about 1:20, about 1:1 to about 1:25, about 1:2 to about 1:25, about 1:5 to about 1:25, about 1:10 to about 1:25, about 1:15 to about 1:25 chamomile:inositol. In some embodiments, chamomile and inositol are present in a mood support composition provided herein at a ratio of about 25:1, about 10:1, about 5:1, about 1:1, 1:5, about 1:10, or about 1:25 chamomile:inositol.

In some embodiments, chamomile and inositol are present in a mood support composition provided herein at a ratio of about 2:1 to about 1:2 chamomile:inositol. For example, chamomile and inositol can be present in a mood support composition provided herein at a ratio of about 2:1, about 1:1, or about 1:2 chamomile:inositol. In some embodiments, chamomile and inositol are present in a mood support composition provided herein at a ratio of about 1:1 chamomile:inositol.

In some embodiments, the lavender and lemon balm together and the chamomile and inositol together are present in a mood support composition provided herein at a ratio of about 25:1 to about 1:1 lavender and lemon balm: chamomile and inositol. For example, the lavender and lemon balm together and the chamomile and inositol together can be present in a mood support composition provided herein at a ratio of about 25:1 to about 20:1, about 25:1 to about 15:1, about 25:1 to about 10:1, about 25:1 to about 5:1, about 25:1 to about 2:1, about 20:1 to about 15:1, about 20:1 to about 10:1, about 20:1 to about 5:1, about 20:1 to about 2:1, 20:1 to about 1:1, about 15:1 to about 10:1, about 15:1 to about 5:1, about 15:1 to about 2:1, about 15:1 to about 1:1, about 10:1 to about 5:1, about 10:1 to about 2:1, about 10:1 to about 1:1, about 5:1 to about 2:1, about 5:1 to about 1:1, or about 2:1 to about 1:1 lavender and lemon balm: chamomile and inositol. In some embodiments, the lavender and lemon balm together and the chamomile and inositol together are present in a mood support composition provided herein at a ratio of about 25:1, about 15:1, about 10:1, about 5:1, about 3:1, about 2:1, or about 1:1 lavender and lemon balm: chamomile and inositol.

In some embodiments, the lavender and lemon balm together and the chamomile and inositol together are present in a mood support composition provided herein at a ratio of about 4:1 to about 1:1 lavender and lemon balm: chamomile and inositol. For example, the lavender and lemon balm together and the chamomile and inositol together can be present in a mood support composition provided herein at a ratio of about 4:1, about 2:1, about 4:3, about 3:1, about 3:2, about 2:1, or about 1:1 lavender and lemon balm: chamomile and inositol. In some embodiments, the lavender and lemon balm together and the chamomile and inositol together are present in a mood support composition provided herein at a ratio of about 3:1 lavender and lemon balm: chamomile and inositol.

In some embodiments, Composition A as provided herein is a composition that includes the ingredients listed in Table 1.

TABLE 1

| | | Amount |
|---|---|---|
| | Amount | (% w/w of |
| Ingredient | (per serving) | Composition A) |
| Lavender | 49 mg to 490 mg | 50% to 80% |
| Lemon Balm | 11 mg to 117 mg | 10% to 20% |
| Chamomile | 9 mg to 98 mg | 5% to 25% |
| Inositol | 9 mg to 98 mg | 5% to 25% |

In some embodiments, the lavender is a lavender extract prepared from flowers of L. angustifolia. In some embodiments, the lemon balm is a lemon balm extract prepared from leaves and stems of M. officinalis. In some embodiments, the chamomile is a chamomile extract prepared from flowers M. chamomilla.

In some embodiments, Composition A as provided herein is a composition that includes the ingredients listed in Table 2.

TABLE 2

| | | Amount |
|---|---|---|
| | | (% w/w of |
| | Amount | Composition |
| Ingredient | (per serving) | A) |
| Lavender | about 75 mg to about 175 mg | 50% to 80% |
| Lemon Balm | about 10 mg to about 80 mg | 10% to 20% |
| Chamomile | about 20 mg to about 70 mg | 5% to 25% |
| Inositol | about 20 mg to about 70 mg | 5% to 25% |
| Total | 205 mg | 100% |

In some embodiments, the lavender is a lavender extract prepared from flowers of L. angustifolia. In some embodiments, the lemon balm is a lemon balm extract prepared from leaves and stems of M. officinalis. In some embodiments, the chamomile is a chamomile extract prepared from flowers M. chamomilla.

### Combinations

Also provided herein is a mood support composition comprising Composition A (e.g., a mood support composition that contains lavender, lemon balm, chamomile, and inositol as provided herein) and one or more other mood support compositions and/or sleep aid compositions. For example, provided herein are mood support compositions comprising Composition A and another mood support composition such as Composition B. In some embodiments, provided herein are mood support compositions comprising Composition A and a sleep aid composition such as Composition C.

In some embodiments, Composition A is present in an amount of about 1% to about 99% w/w of the total composition. For example, Composition A can be present in an amount of about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100% w/w of the total composition. In some embodiments, Composition A is present in an amount of about 5% to about 95% w/w of the total composition.

In some embodiments, a lavender extract is present in an amount of about 0.6% to about 60% w/w of the total composition (e.g., a mood support composition comprising Composition A and one or more other compositions). For example, a lavender extract can be present in an amount of about 0.6% to about 5%, about 0.6% to about 10%, about 0.6% to about 20%, about 0.6% to about 30%, about 0.6% to about 40%, about 0.6% to about 50%, about 5% to about 10%, about 5% to about 20%, about 5% to about 30%, about 5% to about 40%, about 5% to about 50%, about 5% to about 60%, about 10% to about 20%, about 10% to about 30%, about 10% to about 40%, about 10% to about 50%, about 10% to about 60%, about 20% to about 30%, about 20% to about 40%, about 20% to about 50%, about 20% to about 60%, about 30% to about 40%, about 30% to about 50%, about 30% to about 60%, about 40% to about 50%, about 40% to about 60%, or about 50% to about 60% w/w of the total composition. In some embodiments, a lavender extract is present in an amount of about 3% to about 58% w/w of the total composition. In some embodiments, a lavender extract is present in an amount of about 12% to about 19% w/w of the total composition. In some embodiments, a lavender extract is present in an amount of about 12% w/w of the total composition. In some embodiments, a lavender extract is present in an amount of about 19% w/w of the total composition.

In some embodiments, a lemon balm extract is present in an amount of about 0.15% to about 15% w/w of the total composition (e.g., a mood support composition comprising Composition A and one or more other compositions). For example, a lemon balm extract can be present in an amount of about 0.15% to about 1%, about 0.15% to about 3%, about 0.15% to about 5%, about 0.15% to about 8%, about 0.15% to about 10%, about 0.15% to about 12%, about 1% to about 3%, about 1% to about 5%, about 1% to about 8%, about 1% to about 10%, about 1% to about 12%, about 1% to about 15%, about 3% to about 5%, about 3% to about 8%, about 3% to about 10%, about 3% to about 12%, about 3% to about 15%, about 5% to about 8%, about 5% to about 10%, about 5% to about 12%, about 5% to about 15%, about 8% to about 10%, about 8% to about 12%, about 8% to about 15%, about 10% to about 12%, about 10% to about 15%, or about 12% to about 15% w/w of the total composition. In some embodiments, a lemon balm extract is present in an amount of about 0.7% to about 15% w/w of the total composition. In some embodiments, a lemon balm extract is present in an amount of about 2% to about 5% w/w of the total composition. In some embodiments, a lemon balm extract is present in an amount of about 3% w/w of the total composition. In some embodiments, a lemon balm extract is present in an amount of about 4% w/w of the total composition.

In some embodiments, a chamomile extract is present in an amount of about 0.12% to about 12% w/w of the total composition (e.g., a mood support composition comprising Composition A and one or more other compositions). For example, a chamomile extract can be present in an amount of about 0.12% to about 1%, about 0.12% to about 3%, about 0.12% to about 5%, about 0.12% to about 8%, about 0.12% to about 10%, about 1% to about 3%, about 1% to about 5%, about 1% to about 8%, about 1% to about 10%, about 1% to about 12%, about 3% to about 5%, about 3% to about 8%, about 3% to about 10%, about 3% to about 12%, about 5% to about 8%, about 5% to about 10%, about 5% to about 12%, about 8% to about 10%, about 8% to about 12%, or about 10% to about 12% w/w of the total composition. In some embodiments, a chamomile extract is present in an amount of about 2% to about 4% w/w of the total composition. In some embodiments, a chamomile extract is present in an amount of about 2% w/w of the total composition. In some embodiments, a chamomile extract is present in an amount of about 3% w/w of the total composition. In some embodiments, a chamomile extract is present in an amount of about 4% w/w of the total composition.

In some embodiments, inositol is present in an amount of about 0.12% to about 12% w/w of the total composition (e.g., a mood support composition comprising Composition A and one or more other compositions). For example, inositol can be present in an amount of about 0.12% to about 1%, about 0.12% to about 3%, about 0.12% to about 5%, about 0.12% to about 8%, about 0.12% to about 10%, about 1% to about 3%, about 1% to about 5%, about 1% to about 8%, about 1% to about 10%, about 1% to about 12%, about 3% to about 5%, about 3% to about 8%, about 3% to about 10%, about 3% to about 12%, about 5% to about 8%, about 5% to about 10%, about 5% to about 12%, about 8% to about 10%, about 8% to about 12%, or about 10% to about 12% w/w of the total composition. In some embodiments, inositol is present in an amount of about 2% to about 4% w/w of the total composition. In some embodiments, inositol is present in an amount of about 2% w/w of the total composition. In some embodiments, inositol is present in an amount of about 3% w/w of the total composition. In some embodiments, inositol is present in an amount of about 4% w/w of the total composition.

### Composition B

In some embodiments, a mood support composition provided herein can further include one or more of ashwagandha (e.g., an ashwagandha extract), vitamin B12, and folate. Also provided herein are mood support compositions comprising Composition A and Composition B. Composition B is a mood support composition that contains ashwagandha (e.g., an ashwagandha extract), vitamin B12, and optionally folate. In some embodiments, Composition B is a mood support composition that contains ashwagandha (e.g., an ashwagandha extract), vitamin B12, saffron (e.g., a saffron extract), and optionally folate. In some embodiments, Composition B does not comprise saffron.

In some embodiments, Composition B includes saffron. Saffron is a spice derived from the stigmas of Crocus sativus flowers. Saffron can increase the levels of mood-elevating neurotransmitters like serotonin and dopamine. Its ability to modulate neurotransmitters like serotonin, dopamine, and norepinephrine may contribute to its effects on mood. Studies on saffron have shown comparable efficacy to common selective serotonin reuptake inhibitors in reducing depression and anxiety. For improving sleep, studies have shown that saffron can have a beneficial effect. Additionally, saffron has been reported to have antioxidant and anti-inflammatory properties.

Saffron, as used herein, can include saffron threads (e.g., dried whole stigmas), powdered saffron (e.g., ground stigmas), and/or a saffron extract. A saffron extract can be in the form of a liquid (e.g., an aqueous solution), a solid (e.g., a particulate or a powder), or a semi-solid form (e.g., a gel or an emulsion). Any appropriate method can be used to obtain a saffron extract for use in Composition B. For example, a saffron extract can be obtained by aqueous or ethanolic extraction from Crocus sativus stigma.

In some embodiments, saffron is present in an amount of at least about 10 mg per serving of Composition B (e.g., at least about 14 mg, at least about 20 mg, at least about 24 mg, at least about 28 mg, at least about 32 mg, at least about 36 mg, at least about 44 mg, or at least about 50 mg per serving of Composition B). In some embodiments, saffron is present in an amount of no more than about 60 mg per serving of Composition B (e.g., no more than about 50 mg, no more than about 44 mg, no more than about 36 mg, no more than about 32 mg, no more than about 28 mg, or no more than about 24 mg per serving of Composition B).

In some embodiments, a saffron extract is present in an amount of about 14 mg to about 56 mg per serving of Composition B. For example, a saffron extract can be present in an amount of about 14 mg to about 20 mg, about 14 mg to about 26 mg, about 14 mg to about 32 mg, about 14 mg to about 38 mg, about 14 mg to about 44 mg, about 14 mg to about 50 mg, about 20 mg to about 26 mg, about 20 mg to about 32 mg, about 20 mg to about 38 mg, about 20 mg to about 44 mg, about 20 mg to about 50 mg, about 20 mg to about 56 mg, about 32 mg to about 38 mg, about 32 mg to about 44 mg, about 32 mg to about 50 mg, about 32 mg to about 56 mg, about 38 mg to about 44 mg, about 38 mg to about 50 mg, about 38 mg to about 56 mg, about 44 mg to about 50 mg, about 44 mg to about 56 mg, or about 50 mg to about 56 mg per serving of Composition B. In some embodiments, a saffron extract is present in an amount of about 24 mg to about 32 mg per serving of Composition B. In some embodiments, a saffron extract is present in an amount of about 26 mg to about 30 mg per serving of Composition B. In some embodiments, a saffron extract is present in an amount of about 28 mg to about 30 mg per serving of Composition B.

In some embodiments, a saffron extract is present in an amount of about 14 mg, 20 mg, about 22 mg, about 24 mg, about 26 mg, about 28 mg, about 30 mg, about 32 mg, about 34 mg, about 36 mg, about 38 mg, about 44 mg, about 50 mg, or about 56 mg per serving of Composition B. In some embodiments, a saffron extract is present in an amount of about 28 mg per serving of Composition B.

In some embodiments, a saffron extract is present in an amount of about 0.25% to about 30% w/w of the total composition (e.g., Composition A and Composition B combined). For example, a saffron extract can be present in an amount of about 0.25% to about 5%, about 0.25% to about 10%, about 0.25% to about 15%, about 0.25% to about 20%, about 0.25% to about 25%, about 1% to about 5%, about 1% to about 10%, about 1% to about 15%, about 1% to about 20%, about 1% to about 25%, about 1% to about 30%, about 5% to about 10%, about 5% to about 15%, about 5% to about 20%, about 5% to about 25%, about 5% to about 30%, about 10% to about 15%, about 10% to about 20%, about 10% to about 25%, about 10% to about 30%, about 15% to about 20%, about 15% to about 25%, about 15% to about 30%, about 20% to about 25%, about 20% to about 30%, or about 25% to about 30% w/w of the total composition. In some embodiments, a saffron extract is present in an amount of about 1%, about 5%, about 10%, about 15%, about 20%, about 25%, or about 30% w/of the total composition.

Ashwagandha (Withania somnifera) is an evergreen shrub found in tropical and subtropical areas of Asia, Africa, and Europe. It has been traditionally used for reducing stress. Ashwagandha is an adaptogen that can help the body adapt to stress and boost energy, stamina, and endurance. It can support the body's ability to cope with stress by modulating the stress response system and promoting a sense of calm. For example, Ashwagandha can have cortisol-lowering effects and anti-inflammatory properties to help adapt to change and stress. Ashwagandha can also support cognitive function, promote relaxation, and improve sleep quality.

In some embodiments, Composition B can include ashwagandha whole root powder and/or an ashwagandha extract. An ashwagandha extract can be in the form of a liquid (e.g., an aqueous solution), a solid (e.g., a particulate or a powder), or a semi-solid form (e.g., a gel or an emulsion). Any appropriate method can be used to obtain ashwagandha for use in Composition B. For example, ashwagandha whole root powder can be prepared by drying and grinding the root of the ashwagandha plant. As another example, an alcohol (e.g., ethanol) solution can be used to obtain an ashwagandha extract from ashwagandha roots and/or ashwagandha leaves.

In some embodiments, Composition B includes ashwagandha whole root powder. Composition B can contain any appropriate amount of ashwagandha whole root powder. In some embodiments, ashwagandha whole root powder is present in an amount of about 0.25 g to about 6 g per serving of Composition B. For example, ashwagandha whole root powder can be present in an amount of about 0.25 g to about 1 g, about 0.25 g to about 2 g, about 0.25 g to about 3 g, about 0.25 g to about 4 g, about 0.25 g to about 5 g, about 1 g to about 2 g, about 1 g to about 3 g, about 1 g to about 4 g, about 1 g to about 5 g, about 1 g to about 6 g, about 2 g to about 3 g, about 2 g to about 4 g, about 2 g to about 5 g, about 2 g to about 6 g, about 3 g to about 4 g, about 3 g to about 5 g, about 3 g to about 6 g, about 4 g to about 5 g, about 4 g to about 6 g, or about 5 g to about 6 g per serving of Composition B.

In some embodiments, Composition B includes an ashwagandha extract. In some embodiments, an ashwagandha extract is an extract from ashwagandha leaves and/or ashwagandha roots. In some embodiments, an ashwagandha extract is an ashwagandha root extract. For example, an ashwagandha root extract can be an extract where no ashwagandha leaves were used in producing the extract. In some embodiments, the proportions of compounds in an ashwagandha extract are similar to their proportions found naturally (e.g., in ashwagandha leaves and/or ashwagandha roots).

In some embodiments, an ashwagandha extract is present in an amount of at least about 100 mg per serving of Composition B (e.g., at least about 125 mg, at least about 160 mg, at least about 200 mg, at least about 300 mg, at least about 400 mg, at least about 500 mg, at least about 600 mg, or at least about 800 mg per serving of Composition B). In some embodiments, an ashwagandha extract is present in an amount of no more than about 2000 mg per serving of Composition B (e.g., no more than about 1800 mg, no more than about 1600 mg, no more than about 1400 mg, no more than about 1200 mg, no more than about 1000 mg, no more than about 800 mg, or no more than about 600 mg per serving of Composition B).

In some embodiments, an ashwagandha extract is present in an amount of about 125 mg to about 1800 mg per serving of Composition B. For example, an ashwagandha extract can be present in an amount of about 125 mg to about 200 mg, about 125 mg to about 400 mg, about 125 mg to about 600 mg, about 125 mg to about 800 mg, about 125 mg to about 1000 mg, about 125 mg to about 1200 mg, about 125 mg to about 1400 mg, about 125 mg to about 1600 mg, about 200 mg to about 400 mg, about 200 mg to about 600 mg, about 200 mg to about 800 mg, about 200 mg to about 1000 mg, about 200 mg to about 1200 mg, about 200 mg to about 1400 mg, about 200 mg to about 1600 mg, about 200 mg to about 1800 mg, about 400 mg to about 600 mg, about 400 mg to about 800 mg, about 400 mg to about 1000 mg, about 400 mg to about 1200 mg, about 400 mg to about 1400 mg, about 400 mg to about 1600 mg, about 400 mg to about 1800 mg, about 600 mg to about 800 mg, about 600 mg to about 1000 mg, about 600 mg to about 1200 mg, about 600 mg to about 1400 mg, about 600 mg to about 1600 mg, about 600 mg to about 1800 mg, about 800 mg to about 1000 mg, about 800 mg to about 1200 mg, about 800 mg to about 1400 mg, about 800 mg to about 1600 mg, about 800 mg to about 1800 mg, about 1000 mg to about 1200 mg, about 1000 mg to about 1400 mg, about 1000 mg to about 1600 mg, about 1000 mg to about 1800 mg, about 1200 mg to about 1400 mg, about 1200 mg to about 1600 mg, about 1200 mg to about 1800 mg, about 1400 mg to about 1600 mg, about 1400 mg to about 1800 mg, or about 1600 mg to about 1800 mg per serving of Composition B. In some embodiments, an ashwagandha extract is present in an amount of about 300 mg to about 675 mg per serving of Composition B. In some embodiments, an ashwagandha extract is present in an amount of about 525 mg to about 675 mg per serving of Composition B. In some embodiments, an ashwagandha extract is present in an amount of about 550 mg to about 650 mg per serving of Composition B.

In some embodiments, an ashwagandha extract is present in an amount of about 200 mg, 300 mg, about 400 mg, about 500 mg, about 525 mg, about 550 mg, about 575 mg, about 600 mg, about 625 mg, about 650 mg, about 675 mg, about 700 mg, about 800 mg, about 900 mg, about 1000 mg, about 1200 mg, about 1400 mg, about 1600 mg, or about 1800 mg per serving of Composition B. In some embodiments, an ashwagandha extract is present in an amount of about 600 mg per serving of Composition B.

In some embodiments, an ashwagandha extract is present in an amount of about 5% to about 95% w/w of the total composition (e.g., Composition A and Composition B combined). For example, an ashwagandha extract can be present in an amount of about 5% to about 20%, about 5% to about 30%, about 5% to about 40%, about 5% to about 50%, about 5% to about 60%, about 5% to about 70%, about 5% to about 80%, about 5% to about 90%, about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 20% to about 30%, about 20% to about 40%, about 20% to about 50%, about 20% to about 60%, about 20% to about 70%, about 20% to about 80%, about 20% to about 90%, about 20% to about 95%, about 30% to about 40%, about 30% to about 50%, about 30% to about 60%, about 30% to about 70%, about 30% to about 80%, about 30% to about 90%, about 30% to about 95%, about 40% to about 50%, about 40% to about 60%, about 40% to about 70%, about 40% to about 80%, about 40% to about 90%, about 40% to about 95%, about 50% to about 60%, about 50% to about 70%, about 50% to about 80%, about 50% to about 90%, about 50% to about 95%, about 60% to about 70%, about 60% to about 80%, about 60% to about 90%, about 60% to about 95%, about 70% to about 80%, about 70% to about 90%, about 70% to about 95%, about 80% to about 90%, about 80% to about 95%, or about 90% to about 95% w/w of the total composition. In some embodiments, an ashwagandha extract is present in an amount of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% w/w of the total composition.

Ashwagandha whole root powders and ashwagandha extracts can contain one or more withanolides. Withanolides include compounds consisting of a steroid backbone bound to a lactone or a lactone derivative. Non-limiting examples of a withanolide include withaferin A, salpichrolid A, nicandrenone-1, ixocarpalacton A, withaperuvin B, withangulatin C, acnistin I, physalin X, jaborosalactol 18, physalin C, jaborosalactone 7, salpichrolide G, jaborosalactone 37, jaborosalactone 43, subtrifloralactone E, tubonolide A, withametelinone, withaphysalin N, isocarpalactone B, perulactone C, jaborosalactone 15, subtrifloralactone K, taccalonolide A, jaborosalactol 24, physanolide A, exodeconolide A, withanoside VII, 27-hydroxywithanone, dihydrowithaferin A, viscosalactone B, withanoside IV, withanoside V, withaferin A, 12-deoxywithastramonolide, withanolide A, withanone, and withanolide B. In some embodiments, the one or more withanolides include withaferin A.

In some embodiments, ashwagandha whole root powder contains one or more withanolides present in an amount of at least about 0.4%, e.g., at least about 0.5%, at least about 0.6%, at least about 0.7%, at least about 0.8%, at least about 0.9%, at least about 1%, or at least about 1.2%, w/w of the ashwagandha whole root powder. In some embodiments, ashwagandha whole root powder contains one or more withanolides present in an amount of about 0.5% to about 1.5% w/w of the ashwagandha whole root powder. For example, ashwagandha whole root powder can contain one or more withanolides present in an amount of about 0.5% to about 0.8%, about 0.5% to about 1%, about 0.5% to about 1.2%, about 0.8% to about 1%, about 0.8% to about 1.2%, about 0.8% to about 1.5%, about 1% to about 1.2%, about 1% to about 1.5%, or about 1.2% to about 1.5% w/w of the ashwagandha whole root powder.

In some embodiments, an ashwagandha extract contains one or more withanolides present in an amount of at least about 4%, e.g., at least about 5%, at least about 6%, at least about 7%, at least about 8%, at least about 9%, or at least about 10% w/w of the ashwagandha extract. In some embodiments, the ashwagandha extract contains one or more withanolides present in an amount of about 1% to about 12% w/w of the ashwagandha extract. For example, the ashwagandha extract can contain one or more withanolides present in an amount of about 1% to about 2%, about 1% to about 3%, about 1% to about 4%, about 1% to about 5%, about 1% to about 6%, about 1% to about 7%, about 1% to about 8%, about 1% to about 9%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10% w/w of the ashwagandha extract. In some embodiments, the ashwagandha extract contains one or more withanolides present in an amount of about 5% w/w of the ashwagandha extract.

In some embodiments, the one or more withanolides are present in an amount of about 5 mg to about 100 mg per serving of Composition B. For example, the one or more withanolides are present in an amount of about 5 mg to about 10 mg, about 5 mg to 20 mg, about 5 mg to about 30 mg, about 5 mg to about 40 mg, about 5 mg to about 50 mg, about 5 mg to about 60 mg, about 5 mg to about 70 mg, about 5 mg to about 80 mg, about 5 mg to about 90 mg, about 10 mg to 20 mg, about 10 mg to about 30 mg, about 10 mg to about 40 mg, about 10 mg to about 50 mg, about 10 mg to about 60 mg, about 10 mg to about 70 mg, about 10 mg to about 80 mg, about 10 mg to about 90 mg, about 10 mg to about 100 mg, about 20 mg to about 30 mg, about 20 mg to about 40 mg, about 20 mg to about 50 mg, about 20 mg to about 60 mg, about 20 mg to about 70 mg, about 20 mg to about 80 mg, about 20 mg to about 90 mg, about 20 mg to about 100 mg, about 30 mg to about 40 mg, about 30 mg to about 50 mg, about 30 mg to about 60 mg, about 30 mg to about 70 mg, about 30 mg to about 80 mg, about 30 mg to about 90 mg, about 30 mg to about 100 mg, about 40 mg to about 50 mg, about 40 mg to about 60 mg, about 40 mg to about 70 mg, about 40 mg to about 80 mg, about 40 mg to about 90 mg, about 40 mg to about 100 mg, about 50 mg to about 60 mg, about 50 mg to about 70 mg, about 50 mg to about 80 mg, about 50 mg to about 90 mg, about 50 mg to about 100 mg, about 60 mg to about 70 mg, about 60 mg to about 80 mg, about 60 mg to about 90 mg, about 60 mg to about 100 mg, about 70 mg to about 80 mg, about 70 mg to about 90 mg, about 70 mg to about 100 mg, about 80 mg to about 90 mg, about 80 mg to about 100 mg, or about 90 mg to about 100 mg per serving of Composition B. In some embodiments, the one or more withanolides are present in an amount of about 5 mg, 10 mg, 20 mg, about 30 mg, about 40 mg, about 50 mg, about 60 mg, about 70 mg, about 80 mg, about 90 mg, or about 100 mg per serving of Composition B.

In some embodiments, Composition B include a B vitamin, such as vitamin B12. Certain B vitamins, including B12, can play a role in brain function and the production of neurotransmitters involved in mood regulation. Vitamin B12 includes hydroxocobalamin, cyanocobalamin, methylcobalamin, and adenosylcobalamin. Forms of vitamin B12 such as methylcobalamin, adenosylcobalamin, and hydroxycobalamin can have superior bioavailability compared to cyanocobalamin. See, e.g., Paul and Brady, Integr. Med. (Encinitas). 16 (1): 42-49 (2017). Any appropriate method can be used to obtain a vitamin B12 for use in Composition B. For example, a vitamin B12 can be obtained by chemical synthesis.

In some embodiments, vitamin B12 is present in an amount of at least about 0.4 μg per serving of Composition B (e.g., at least about 0.5 μg, at least about 1 μg, at least about 5 μg, at least about 10 μg, at least about 20 μg, at least about 30 μg, at least about 40 μg, at least about 50 μg, or at least about 60 μg per serving of Composition B). In some embodiments, vitamin B12 is present in an amount of no more than about 120 μg per serving of Composition B (e.g., no more than about 100 μg, no more than about 90 μg, no more than about 80 μg, no more than about 70 μg, no more than about 60 μg, no more than about 50 μg, no more than about 40 μg, no more than about 30 μg, or no more than about 20 μg per serving of Composition B).

In some embodiments, vitamin B12 is present in an amount of about 0.5 μg to about 100 μg per serving of Composition B. For example, vitamin B12 can be present in an amount of about 0.5 μg to about 1 μg, about 0.5 μg to about 5 μg, about 0.5 μg to about 10 μg, about 0.5 μg to about 20 μg, about 0.5 μg to about 30 μg, about 0.5 μg to about 40 μg, about 0.5 μg to about 50 μg, about 0.5 μg to about 60 μg, about 0.5 μg to about 70 μg, about 0.5 μg to about 80 μg, about 0.5 μg to about 90 μg, about 1 μg to about 5 μg, about 1 μg to about 10 μg, about 1 μg to about 20 μg, about 1 μg to about 30 μg, about 1 μg to about 40 μg, about 1 μg to about 50 μg, about 1 μg to about 60 μg, about 1 μg to about 70 μg, about 1 μg to about 80 μg, about 1 μg to about 90 μg, about 1 μg to about 100 μg, about 5 μg to about 10 μg, about 5 μg to about 20 μg, about 5 μg to about 30 μg, about 5 μg to about 40 μg, about 5 μg to about 50 μg, about 5 μg to about 60 μg, about 5 μg to about 70 μg, about 5 μg to about 80 μg, about 5 μg to about 90 μg, about 5 μg to about 100 μg, about 10 μg to about 20 μg, about 10 μg to about 30 μg, about 10 μg to about 40 μg, about 10 μg to about 50 μg, about 10 μg to about 60 μg, about 10 μg to about 70 μg, about 10 μg to about 80 μg, about 10 μg to about 90 μg, about 10 μg to about 100 μg, about 20 μg to about 30 μg, about 20 μg to about 40 μg, about 20 μg to about 50 μg, about 20 μg to about 60 μg, about 20 μg to about 70 μg, about 20 μg to about 80 μg, about 20 μg to about 90 μg, about 20 μg to about 100 μg, about 30 μg to about 40 μg, about 30 μg to about 50 μg, about 30 μg to about 60 μg, about 30 μg to about 70 μg, about 30 μg to about 80 μg, about 30 μg to about 90 μg, about 30 μg to about 100 μg, about 40 μg to about 50 μg, about 40 μg to about 60 μg, about 40 μg to about 70 μg, about 40 μg to about 80 μg, about 40 μg to about 90 μg, about 40 μg to about 100 μg, about 50 μg to about 60 μg, about 50 μg to about 70 μg, about 50 μg to about 80 μg, about 50 μg to about 90 μg, about 50 μg to about 100 μg, about 60 μg to about 70 μg, about 60 μg to about 80 μg, about 60 μg to about 90 μg, about 60 μg to about 100 μg, about 70 μg to about 80 μg, about 70 μg to about 90 μg, about 70 μg to about 100 μg, about 80 μg to about 90 μg, about 80 μg to about 100 μg, or about 90 μg to about 100 μg per serving of Composition B. In some embodiments, vitamin B12 is present in an amount of about 3 μg to about 60 μg per serving of Composition B. In some embodiments, vitamin B12 is present in an amount of about 2 μg to about 10 μg per serving of Composition B. In some embodiments, vitamin B12 is present in an amount of about 4 μg to about 8 μg per serving of Composition B.

In some embodiments, vitamin B12 is present in an amount of 0.5 μg, 1 μg, about 2 μg, about 3 μg, about 4 μg, about 5 μg, about 6 μg, about 7 μg, about 8 μg, about 9 μg, about 10 μg, about 20 μg, about 30 μg, about 40 μg, about 50 μg, about 60 μg, about 80 μg, about 100 μg per serving of Composition B. In some embodiments, vitamin B12 is present in an amount of about 6 μg per serving of Composition B.

In some embodiments, vitamin B12 is present in an amount of about 0.00001% to about 0.5% w/w of the total composition (e.g., Composition A and Composition B combined). For example, vitamin B12 can be present in an amount of about 0.00001% to about 0.0005%, about 0.00001% to about 0.001%, about 0.00001% to about 0.005%, about 0.00001% to about 0.01%, about 0.00001% to about 0.05%, about 0.00001% to about 0.1%, about 0.00005% to about 0.0005%, about 0.00005% to about 0.001%, about 0.00005% to about 0.005%, about 0.00005% to about 0.01%, about 0.00005% to about 0.05%, about 0.00005% to about 0.1%, about 0.00005% to about 0.5%, about 0.0001% to about 0.0005%, about 0.0001% to about 0.001%, about 0.0001% to about 0.005%, about 0.0001% to about 0.01%, about 0.0001% to about 0.05%, about 0.0001% to about 0.1%, about 0.0001% to about 0.5%, about 0.0005% to about 0.001%, about 0.0005% to about 0.005%, about 0.0005% to about 0.01%, about 0.0005% to about 0.05%, about 0.0005% to about 0.1%, about 0.0005% to about 0.5%, about 0.001% to about 0.005%, about 0.001% to about 0.01%, about 0.001% to about 0.05%, about 0.001% to about 0.1%, about 0.001% to about 0.5%, about 0.005% to about 0.01%, about 0.005% to about 0.05%, about 0.005% to about 0.1%, about 0.005% to about 0.5%, about 0.01% to about 0.05%, about 0.01% to about 0.1%, about 0.01% to about 0.5%, about 0.05% to about 0.1%, about 0.05% to about 0.5%, or about 0.1% to about 0.5% w/w of the total composition. In some embodiments, vitamin B12 is present in an amount of about 0.0001%, about 0.0005%, about 0.001%, about 0.005%, about 0.01%, about 0.05%, about 0.1%, about 0.5% w/w of the total composition.

In some embodiments, the vitamin B12 is methylcobalamin.

Composition B can further include folate. Folate (also known as vitamin B9) refers to folates naturally found in foods and folates used in dietary supplements and fortified foods such as folic acid. Non-limiting examples of folate include folic acid, tetrahydrofolic acid, 5-methyltetrahydrofolate (also known as 5-MTHF, 5-methyl-folate, and methylfolate) or a salt thereof, a methenyltetrahydrofolate, folinic acid, folacin, and pteroylglutamic acid. Non-limiting examples of salts of 5-methyltetrahydrofolate include calcium L-5-methyltetrahydrofolate and sodium L-5-methyltetrahydrofolate. Any appropriate method can be used to obtain folate for use Composition B. For example, folates can be obtained by chemical synthesis.

In some embodiments, folate is present in an amount of at least about 0.05 mg per serving of Composition B (e.g., at least about 0.1 mg, at least about 0.2 mg, at least about 0.4 mg, at least about 0.6 mg, at least about 0.8 mg, at least about 1 mg, at least about 1.4 mg, or at least about 1.6 mg per serving of Composition B). In some embodiments, folate is present in an amount of no more than about 2.2 mg per serving of Composition B (e.g., no more than about 2 mg, no more than about 1.8 mg, no more than about 1.6 mg, no more than about 1.4 mg, no more than about 1 mg, no more than about 0.8 mg, no more than about 0.6 mg, or no more than about 0.4 mg per serving of Composition B).

In some embodiments, folate is present in an amount of about 0.1 mg to about 2 mg per serving of Composition B. For example, folate can be present in an amount of about 0.1 mg to about 0.2 mg, about 0.1 mg to about 0.4 mg, about 0.1 mg to about 0.6 mg, about 0.1 mg to about 0.8 mg, about 0.1 mg to about 1 mg, about 0.1 mg to about 1.2 mg, about 0.1 mg to about 1.4 mg, about 0.1 mg to about 1.6 mg, about 0.1 mg to about 1.8 mg, about 0.2 mg to about 0.4 mg, about 0.2 mg to about 0.6 mg, about 0.2 mg to about 0.8 mg, about 0.2 mg to about 1 mg, about 0.2 mg to about 1.2 mg, about 0.2 mg to about 1.4 mg, about 0.2 mg to about 1.6 mg, about 0.2 mg to about 1.8 mg, about 0.2 mg to about 2 mg, about 0.4 mg to about 0.6 mg, about 0.4 mg to about 0.8 mg, about 0.4 mg to about 1 mg, about 0.4 mg to about 1.2 mg, about 0.4 mg to about 1.4 mg, about 0.4 mg to about 1.6 mg, about 0.4 mg to about 1.8 mg, about 0.4 mg to about 2 mg, about 0.6 mg to about 0.8 mg, about 0.6 mg to about 1 mg, about 0.6 mg to about 1.2 mg, about 0.6 mg to about 1.4 mg, about 0.6 mg to about 1.6 mg, about 0.6 mg to about 1.8 mg, about 0.6 mg to about 2 mg, about 0.8 mg to about 1 mg, about 0.8 mg to about 1.2 mg, about 0.8 mg to about 1.4 mg, about 0.8 mg to about 1.6 mg, about 0.8 mg to about 1.8 mg, about 0.8 mg to about 2 mg, about 1 mg to about 1.2 mg, about 1 mg to about 1.4 mg, about 1 mg to about 1.6 mg, about 1 mg to about 1.8 mg, about 1 mg to about 2 mg, about 1.2 mg to about 1.4 mg, about 1.2 mg to about 1.6 mg, about 1.2 mg to about 1.8 mg, about 1.2 mg to about 2 mg, about 1.4 mg to about 1.6 mg, about 1.4 mg to about 1.4 mg, about 1.2 mg to about 2 mg, about 1.6 mg to about 1.4 mg, about 1.6 mg to about 2 mg, or about 1.8 mg to about 2 mg per serving of Composition B. In some embodiments, folate is present in an amount of about 0.2 mg to about 0.8 mg per serving of Composition B.

In some embodiments, folate is present in an amount of about 0.2 mg, about 0.4 mg, about 0.6 mg, about 0.8 mg, about 1 mg, about 1.4 mg, or about 2 mg per serving of Composition B. In some embodiments, folate is present in an amount of about 0.4 mg per serving of Composition B.

In some embodiments, the amount of folate in mg is the amount of folate as mg of dietary folate equivalents (DFEs). In some embodiments, 1 mg DFE is equal to 1 mg food folate. In some embodiments, 1 mg DFE is equal to 0.6 mg folic acid from fortified foods or dietary supplements consumed with foods. In some embodiments, 1 mg DFE is equal to 0.5 mg folic acid from dietary supplements taken on an empty stomach. In some embodiments, 1 mg DFE is equal to 0.59 mg of a methyltetrahydrofolate. For example, if Composition B contains 0.4 mg DFE of a methyltetrahydrofolate, it contains 0.235 mg of the methyltetrahydrofolate.

In some embodiments, folate is present in an amount of about 0.0001% to about 1% w/w of the total composition (e.g., Composition A and Composition B combined). For example, folate can be present in an amount of about 0.0001% to about 0.005%, about 0.0001% to about 0.01%, about 0.0001% to about 0.05%, about 0.0001% to about 0.1%, about 0.0001% to about 0.5%, about 0.0005% to about 0.005%, about 0.0005% to about 0.01%, about 0.0005% to about 0.05%, about 0.0005% to about 0.1%, about 0.0005% to about 0.5%, about 0.0005% to about 1%, about 0.001% to about 0.005%, about 0.001% to about 0.01%, about 0.001% to about 0.05%, about 0.001% to about 0.1%, about 0.001% to about 0.5%, about 0.001% to about 1%, about 0.005% to about 0.01%, about 0.005% to about 0.05%, about 0.005% to about 0.1%, about 0.005% to about 0.5%, about 0.005% to about 1%, about 0.01% to about 0.05%, about 0.01% to about 0.1%, about 0.01% to about 0.5%, about 0.01% to about 1%, about 0.05% to about 0.1%, about 0.05% to about 0.5%, about 0.05% to about 1%, about 0.1% to about 0.5%, about 0.1% to about 1%, or about 0.5% to about 1% w/w of the total composition. In some embodiments, folate is present in an amount of about 0.001%, about 0.005%, about 0.01%, about 0.05%, about 0.1%, about 0.5%, or about 1% w/w of the total composition.

In some embodiments, the folate is calcium methyltetrahydrofolate.

In some embodiments, a mood support composition comprising Composition A and Composition B is a composition that includes lavender (e.g., a lavender extract), lemon balm (e.g., a lemon balm extract), chamomile (e.g., a chamomile extract), inositol, ashwagandha, vitamin B12, and folate.

In some embodiments, a mood support composition provided herein is a composition that includes the ingredients listed in Table 3.

TABLE 3

| | Amount |
|---|---|
| Ingredient | (per serving) |
| Lavender | 49 mg to 490 mg |
| Lemon Balm | 11 mg to 117 mg |
| Chamomile | 9 mg to 98 mg |
| Inositol | 9 mg to 98 mg |
| Ashwagandha extract | 300 mg to 675 mg |
| Vitamin B12 | 3 μg to 60 μg |
| Folate | 200 μg to 800 μg |

In some embodiments, a mood support composition provided herein is a composition that includes the ingredients listed in Table 4.

TABLE 4

| | Amount | |
|---|---|---|
| Ingredient | (per serving) | |
| Lavender | about 75 mg to about 175 mg | |
| Lemon Balm | about 10 mg to about 80 mg | |
| Chamomile | about 20 mg to about 70 mg | |
| Inositol | about 20 mg to about 70 mg | |
| Ashwagandha extract | 600 | mg |
| Vitamin B12 | 6 | μg |
| Folate | 400 | μg |

In some embodiments, the lavender is a lavender extract prepared from flowers of L. angustifolia. In some embodiments, the lemon balm is a lemon balm extract prepared from leaves and stems of M. officinalis. In some embodiments, the chamomile is a chamomile extract prepared from flowers M. chamomilla.

In some embodiments, the ashwagandha extract contains at least about 5% withanolides. In some embodiments, the ashwagandha extract contains about 5% withanolides. In some embodiments, the ashwagandha extract is an ashwagandha root extract. In some embodiments, the vitamin B12 is methylcobalamin. In some embodiments, the folate is calcium methyltetrahydrofolate.

In some embodiments, a mood support composition comprising Composition A and Composition B is a composition that includes the ingredients listed in Table 5.

TABLE 5

| | Amount |
|---|---|
| Ingredient | (per serving) |
| Lavender | 49 mg to 490 mg |
| Lemon Balm | 11 mg to 117 mg |
| Chamomile | 9 mg to 98 mg |
| Inositol | 9 mg to 98 mg |
| Saffron extract | 28 mg to 30 mg |
| Ashwagandha extract | 300 mg to 675 mg |
| Vitamin B12 | 3 μg to 60 μg |
| Folate | 200 μg to 800 μg |

In some embodiments, a mood support composition comprising Composition A and Composition B is a composition that includes the ingredients listed in Table 6.

TABLE 6

| | Amount | |
|---|---|---|
| Ingredient | (per serving) | |
| Lavender | about 75 mg to about 175 mg | |
| Lemon Balm | about 10 mg to about 80 mg | |
| Chamomile | about 20 mg to about 70 mg | |
| Inositol | about 20 mg to about 70 mg | |
| Saffron extract | 28 | mg |
| Ashwagandha extract | 600 | mg |
| Vitamin B12 | 6 | μg |
| Folate | 400 | μg |

In some embodiments, the lavender is a lavender extract prepared from flowers of L. angustifolia. In some embodiments, the lemon balm is a lemon balm extract prepared from leaves and stems of M. officinalis. In some embodiments, the chamomile is a chamomile extract prepared from flowers M. chamomilla.

In some embodiments, the ashwagandha extract contains at least about 5% withanolides. In some embodiments, the ashwagandha extract contains about 5% withanolides. In some embodiments, the ashwagandha extract is an ashwagandha root extract. In some embodiments, the vitamin B12 is methylcobalamin. In some embodiments, the folate is calcium methyltetrahydrofolate.

### Composition C

Provided herein are mood support compositions comprising Composition A and Composition C. Composition C is a sleep aid composition that contains melatonin, L-theanine, GABA (gamma-aminobutyric acid), and an ashwagandha extract. In some embodiments, Composition C further contains a valerian root extract.

Melatonin is a hormone that can be produced by the pineal gland in the brain to help regulate sleep cycles. In some embodiments, melatonin is present in an amount of about 0.1 mg to about 10 mg per serving of Composition C. For example, melatonin can be present in an amount of about 0.1 mg to about 1 mg, about 0.1 mg to about 2 mg, about 0.1 mg to about 3 mg, about 0.1 mg to about 4 mg, about 0.1 mg to about 5 mg, about 0.1 mg to about 6 mg, about 0.1 mg to about 7 mg, about 0.1 mg to about 8 mg, about 0.1 mg to about 9 mg, about 1 mg to about 2 mg, about 1 mg to about 3 mg, about 1 mg to about 4 mg, about 1 mg to about 5 mg, about 1 mg to about 6 mg, about 1 mg to about 7 mg, about 1 mg to about 8 mg, about 1 mg to about 9 mg, about 2 mg to about 3 mg, about 2 mg to about 4 mg, about 2 mg to about 5 mg, about 2 mg to about 6 mg, about 2 mg to about 7 mg, about 2 mg to about 8 mg, about 2 mg to about 9 mg, about 2 mg to about 10 mg, about 3 mg to about 4 mg, about 3 mg to about 5 mg, about 3 mg to about 6 mg, about 3 mg to about 7 mg, about 3 mg to about 8 mg, about 3 mg to about 9 mg, about 3 mg to about 10 mg, about 4 mg to about 5 mg, about 4 mg to about 6 mg, about 4 mg to about 7 mg, about 4 mg to about 8 mg, about 4 mg to about 9 mg, about 4 mg to about 10 mg, about 5 mg to about 6 mg, about 5 mg to about 7 mg, about 5 mg to about 8 mg, about 5 mg to about 9 mg, about 5 mg to about 10 mg, about 6 mg to about 7 mg, about 6 mg to about 8 mg, about 6 mg to about 9 mg, about 6 mg to about 10 mg, about 7 mg to about 8 mg, about 7 mg to about 9 mg, about 7 mg to about 10 mg, about 8 mg to about 9 mg, about 8 mg to about 10 mg, or about 9 mg to about 10 mg per serving of Composition C. In some embodiments, the melatonin is present in an amount of about 0.1 mg to about 5 mg per serving of Composition C. In some embodiments, the melatonin is present in an amount of about 0.1 mg, about 1 mg, about 2 mg, about 3 mg, about 4 mg, about 5 mg, about 6 mg, about 7 mg, about 8 mg, about 9 mg, or about 10 mg per serving of Composition C.

In some embodiments, melatonin is present in an amount of about 0.25% to about 1% w/w of the total composition (e.g., Composition A and Composition C combined). For example, melatonin can be present in an amount of about 0.25% to about 0.3%, about 0.25% to about 0.4%, about 0.25% to about 0.5%, about 0.25% to about 0.6%, about 0.25% to about 0.7%, about 0.25% to about 0.8%, about 0.25% to about 0.9%, about 0.3% to about 0.4%, about 0.3% to about 0.5%, about 0.3% to about 0.6%, about 0.3% to about 0.7%, about 0.3% to about 0.8%, about 0.3% to about 0.9%, about 0.3% to about 1%, about 0.4% to about 0.5%, about 0.4% to about 0.6%, about 0.4% to about 0.7%, about 0.4% to about 0.8%, about 0.4% to about 0.9%, about 0.4% to about 1%, about 0.5% to about 0.6%, about 0.5% to about 0.7%, about 0.5% to about 0.8%, about 0.5% to about 0.9%, about 0.5% to about 1%, about 0.6% to about 0.7%, about 0.6% to about 0.8%, about 0.6% to about 0.9%, about 0.6% to about 1%, about 0.7% to about 0.8%, about 0.7% to about 0.9%, about 0.7% to about 1%, about 0.8% to about 0.9%, about 0.8% to about 1%, or about 0.9% to about 1% w/w of the total composition. In some embodiments, melatonin is present in an amount of about 0.5% to about 0.75% w/w of the total composition. In some embodiments, melatonin is present in an amount of about 0.25%, about 0.3%, about 0.4%, about 0.5%, about 0.6%, about 0.7%, about 0.8%, about 0.9%, or about 1% w/w of the total composition.

In some embodiments, Composition C can be formulated for rapid release of melatonin, sustained release of melatonin, or a combination thereof. For example, when Composition C is formulated for rapid release of melatonin, the melatonin can be released within a period of time (e.g., a first period of time) after taking Composition C. In some embodiments, when Composition C is formulated for sustained release of melatonin, the melatonin can be released gradually over a period of time (e.g., a second period of time) that is longer than the period of time for a rapid release formulation.

In some embodiments, the period of time for the release of melatonin in a rapid release formulation (e.g., the first period of time) is about 1 hour or less, e.g., about 1 hour, about 45 minutes, about 30 minutes, or about 15 minutes. In some embodiments, the period of time for the release of melatonin in a rapid release formulation (e.g., the first period of time) is about 1 hour. In some embodiments, the period of time for the release of melatonin in a sustained release formulation (e.g., the second period of time) is about 3 or more hours, e.g., about 3 hours, about 4 hours, about 5 hours, about 6 hours, about 7 hours, or about 8 hours. In some embodiments, the period of time for the release of melatonin in a sustained release formulation (e.g., the second period of time) is about 6 hours. In some embodiments, a second period of time begins when the first period of time ends.

In some embodiments, Composition C is formulated for a combination of rapid release and sustained release of melatonin. For example, a portion of the melatonin in Composition C can be released within a first period of time, and all or most of the remaining melatonin in Composition C can be released over a second period of time. In some embodiments, about 20% to about 60% w/w of the melatonin in Composition C is released within a first period of time. For example, about 20% to about 30%, about 20% to about 40%, about 20% to about 50%, about 30% to about 40%, about 30% to about 50%, about 30% to about 40%, or about 40% to about 50% w/w of the melatonin in Composition C can be released within a first period of time. In some embodiments, about 40% to about 80% w/w of the melatonin in Composition C is released over a second period of time. For example, about 40% to about 50%, about 40% to about 60%, about 40% to about 70%, about 50% to about 60%, about 50% to about 70%, about 50% to about 80%, about 60% to about 70%, about 60% to about 80%, or about 70% to about 80% w/w of the melatonin in Composition C can be released over a second period of time.

In some embodiments, about 30% to about 40% w/w of the melatonin in Composition C is released within a first period of time (e.g., about 1 hour) and about 50% to about 60% w/w of the melatonin in Composition C is released over a second period of time (e.g., about 50% to about 60% w/w of the melatonin is released over about 6 hours following the first period of time). In some embodiments, about 30% to about 40% w/w of the melatonin in Composition C is released within a first period of time (e.g., about 1 hour) and about 50% to about 60% w/w of the melatonin in Composition C is released over a second period of time (e.g., about 50% to about 60% w/w of the melatonin is released over about 7 hours following the first period of time). In some embodiments, about 40% w/w of the melatonin in Composition C is released within a first period of time (e.g., about 1 hour) and about 60% w/w of the melatonin in Composition C is released over a second period of time (e.g., about 60% w/w of the melatonin is released over about 6 hours following the first period of time). In some embodiments, about 40% w/w of the melatonin in Composition C is released within a first period of time (e.g., about 1 hour) and about 60% w/w of the melatonin in Composition C is released over a second period of time (e.g., about 60% w/w of the melatonin is released over about 7 hours following the first period of time).

L-theanine is an amino acid commonly found in tea leaves. In some embodiments, L-theanine is present in an amount of about 25 mg to about 500 mg per serving of Composition C. For example, L-theanine can be present in an amount of about 25 mg to about 100 mg, about 25 mg to about 200 mg, about 25 mg to about 300 mg, about 25 mg to about 400 mg, about 50 mg to about 100 mg, about 50 mg to about 200 mg, about 50 mg to about 300 mg, about 50 mg to about 400 mg, about 50 mg to about 500 mg, about 100 mg to about 200 mg, about 100 mg to about 300 mg, about 100 mg to about 400 mg, about 100 mg to about 500 mg, about 200 mg to about 300 mg, about 200 mg to about 400 mg, about 200 mg to about 500 mg, about 300 mg to about 400 mg, about 300 mg to about 500 mg, or about 400 mg to about 500 mg per serving of Composition C. In some embodiments, L-theanine is present in an amount of about 100 mg to about 300 mg per serving of Composition C. In some embodiments, L-theanine is present in an amount of about 150 mg to about 500 mg per serving of Composition C. In some embodiments, L-theanine is present in an amount of about 150 mg to about 300 mg per serving of Composition C. In some embodiments, L-theanine is present in an amount of about 150 mg to about 250 mg per serving of Composition C. In some embodiments, L-theanine is present in an amount of about 175 mg to about 225 mg per serving of Composition C.

In some embodiments, L-theanine is present in an amount of about 50 mg, about 100 mg, about 200 mg, about 300 mg, about 400 mg, or about 500 mg per serving of Composition C. In some embodiments, L-theanine is present in an amount of about 200 mg per serving of Composition C.

In some embodiments, L-theanine is present in an amount of about 6% to about 25% w/w of the total composition (e.g., Composition A and Composition C combined). For example, L-theanine can be present in an amount of about 6% to about 10%, about 6% to about 15%, about 6% to about 20%, about 10% to about 15%, about 10% to about 20%, about 10% to about 25%, about 15% to about 20%, about 15% to about 25%, or about 20% to about 25% w/w of the total composition. In some embodiments, L-theanine is present in an amount of about 9% to about 11% w/w of the total composition In some embodiments, L-theanine is present in an amount of about 6%, about 10%, about 15%, about 19%, about 20%, about 21%, or about 25% w/w of the total composition.

GABA is a neurotransmitter that can, for example, promote relaxation, have a calming effect on the nervous system, help regulate sleep cycles, and/or improve sleep. In some embodiments, GABA is present in an amount of about 100 mg to about 1000 mg per serving of Composition C. For example, GABA can be present in an amount of about 100 mg to about 200 mg, about 100 mg to about 400 mg, about 100 mg to about 600 mg, about 100 mg to about 800 mg, 200 mg to about 400 mg, about 200 mg to about 600 mg, about 200 mg to about 800 mg, about 200 mg to about 1000 mg, about 400 mg to about 600 mg, about 400 mg to about 800 mg, about 400 mg to about 1000 mg, about 600 mg to about 800 mg, about 600 mg to about 1000 mg, or about 800 mg to about 1000 mg per serving of Composition C. In some embodiments, GABA is present in an amount of about 100 mg to about 500 mg per serving of Composition C. In some embodiments, GABA is present in an amount of about 200 mg to about 600 mg per serving of Composition C. In some embodiments, GABA is present in an amount of about 150 mg to about 450 mg per serving of Composition C. In some embodiments, GABA is present in an amount of about 200 mg to about 400 mg per serving of Composition C. In some embodiments, GABA is present in an amount of about 250 mg to about 350 mg per serving of Composition C. In some embodiments, GABA is present in an amount of about 275 mg to about 325 mg per serving of Composition C.

In some embodiments, GABA is present in an amount of about 100 mg, about 200 mg, about 250 mg, about 300 mg, about 350 mg, about 400 mg, about 600 mg, about 800 mg, or about 1000 mg per serving of Composition C. In some embodiments, GABA is present in an amount of about 300 mg per serving of Composition C.

In some embodiments, GABA is present in an amount of about 10% to about 34% w/w of the total composition (e.g., Composition A and Composition C combined). For example, GABA can be present in an amount of about 10% to about 15%, about 10% to about 20%, about 10% to about 25%, about 10% to about 30%, about 15% to about 20%, about 15% to about 25%, about 15% to about 30%, about 15% to about 34%, about 20% to about 25%, about 20% to about 30%, about 20% to about 34%, or about 30% to about 34% w/w of the total composition. In some embodiments, GABA is present in an amount of about 13% to about 18% w/w of the total composition. In some embodiments, GABA is present in an amount of about 10%, about 15%, about 20%, about 25%, about 28%, about 30%, about 32%, or about 34% w/w of the total composition.

In some embodiments, Composition C can include ashwagandha whole root powder and/or an ashwagandha extract. In some embodiments, Composition C includes ashwagandha whole root powder. In some embodiments, ashwagandha whole root powder is present in an amount of about 2 g to about 6 g (e.g., about 2 g to about 3 g, about 2 g to about 4 g, about 2 g to about 5 g, about 3 g to about 4 g, about 3 g to about 5 g, about 3 g to about 6 g, about 4 g to about 5 g, about 4 g to about 6 g, or about 5 g to about 6 g) per serving of Composition C.

In some embodiments, Composition C includes an ashwagandha extract. In some embodiments, an ashwagandha extract is an extract from ashwagandha leaves and/or ashwagandha roots (e.g., any of the ashwagandha extracts described herein).

In some embodiments, an ashwagandha extract is present in an amount of about 50 mg to about 1800 mg per serving of Composition C. For example, about 50 mg to about 100 mg, about 50 mg to about 150 mg, about 50 mg to about 200 mg, about 50 mg to about 250 mg, about 50 mg to about 300 mg, about 50 mg to about 350 mg, about 50 mg to about 400 mg, about 50 mg to about 450 mg, about 50 mg to about 500 mg, about 50 mg to about 550 mg, about 50 mg to about 600 mg, about 50 mg to about 800 mg, about 50 mg to about 1000 mg, about 50 mg to about 1400 mg, about 100 mg to about 150 mg, about 100 mg to about 200 mg, about 100 mg to about 250 mg, about 100 mg to about 300 mg, about 100 mg to about 350 mg, about 100 mg to about 400 mg, about 100 mg to about 450 mg, about 100 mg to about 500 mg, about 100 mg to about 550 mg, about 100 mg to about 600 mg, about 100 mg to about 800 mg, about 100 mg to about 1000 mg, about 100 mg to about 1400 mg, about 100 mg to about 1800 mg, about 200 mg to about 250 mg, about 200 mg to about 300 mg, about 200 mg to about 350 mg, about 200 mg to about 400 mg, about 200 mg to about 450 mg, about 200 mg to about 500 mg, about 200 mg to about 550 mg, about 200 mg to about 600 mg, about 200 mg to about 800 mg, about 200 mg to about 2000 mg, about 200 mg to about 1400 mg, about 200 mg to about 1800 mg, about 300 mg to about 350 mg, about 300 mg to about 400 mg, about 300 mg to about 450 mg, about 300 mg to about 500 mg, about 300 mg to about 550 mg, about 300 mg to about 600 mg, about 300 mg to about 800 mg, about 300 mg to about 1000 mg, about 300 mg to about 1400 mg, about 300 mg to about 1800 mg, about 400 mg to about 450 mg, about 400 mg to about 500 mg, about 400 mg to about 550 mg, about 400 mg to about 600 mg, about 400 mg to about 800 mg, about 400 mg to about 1000 mg, about 400 mg to about 1400 mg, about 400 mg to about 1800 mg, about 500 mg to about 550 mg, about 500 mg to about 600 mg, about 500 mg to about 800 mg, about 500 mg to about 1000 mg, about 500 mg to about 1400 mg, about 500 mg to about 1800 mg, about 600 mg to about 800 mg, about 600 mg to about 1000 mg, about 600 mg to about 1400 mg, about 600 mg to about 1800 mg, about 800 mg to about 1000 mg, about 800 mg to about 1400 mg, about 800 mg to about 1800 mg, about 1000 mg to about 1400 mg, about 1000 mg to about 1800 mg, or about 1400 mg to about 1800 mg per serving of Composition C. In some embodiments, an ashwagandha extract is present in an amount of about 125 mg to about 675 mg per serving of Composition C.

In some embodiments, an ashwagandha extract is present in an amount of about 100 mg, about 125 mg, about 130 mg, about 135 mg, about 140 mg, about 150 mg, about 175 mg, about 200 mg, about 250 mg, about 300 mg, about 350 mg, about 400 mg, about 450 mg, or about 500 mg per serving of Composition C. In some embodiments, an ashwagandha extract is present in an amount of about 125 mg per serving of Composition C. In some embodiments, an ashwagandha extract is present in an amount of about 128 mg per serving of Composition C.

In some embodiments, an ashwagandha extract is present in an amount of about 10% to about 40% w/w of the total composition (e.g., Composition A and Composition C combined). For example, an ashwagandha extract can be present in an amount of about 10% to about 15%, about 10% to about 20%, about 10% to about 25%, about 10% to about 30%, about 10% to about 35%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 30% to about 35%, about 30% to about 40%, about 35% to about 40% w/w of the total composition. In some embodiments, an ashwagandha extract is present in an amount of about 20% to about 25% w/w of the total composition. In some embodiments, an ashwagandha extract is present in an amount of about 10%, about 12%, about 15%, about 20%, about 25%, about 28%, about 30%, about 35%, or about 40% w/w of the total composition.

In some embodiments, ashwagandha whole root powder contains one or more withanolides present in an amount of at least about 0.4%, e.g., at least about 0.5%, at least about 0.6%, at least about 0.7%, at least about 0.8%, at least about 0.9%, at least about 1%, or at least about 1.2%, w/w of the ashwagandha whole root powder.

In some embodiments, an ashwagandha extract contains one or more withanolides present in an amount of at least about 4%, e.g., at least about 5%, at least about 6%, at least about 7%, at least about 8%, at least about 9%, or at least about 10% w/w of the ashwagandha extract. In some embodiments, the ashwagandha extract contains one or more withanolides present in an amount of about 1% to about 12% w/w of the ashwagandha extract. For example, the ashwagandha extract can contain one or more withanolides present in an amount of about 1% to about 2%, about 1% to about 3%, about 1% to about 4%, about 1% to about 5%, about 1% to about 6%, about 1% to about 7%, about 1% to about 8%, about 1% to about 9%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10%, about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10% w/w of the ashwagandha extract. In some embodiments, the ashwagandha extract contains one or more withanolides present in an amount of about 5% w/w of the ashwagandha extract.

In some embodiments, Composition C further includes a valerian root extract. Valerian (Valeriana officinalis) is a perennial plant native to Europe and Asia and naturalized in North America. A valerian root extract can be in the form of a liquid (e.g., an aqueous solution), a solid (e.g., a particulate or a powder), or a semi-solid form (e.g., a gel or an emulsion). Any appropriate method can be used to obtain a valerian root extract for use in Composition C. For example, an aqueous solution can be used to obtain a valerian root extract from valerian.

In some embodiments, a valerian root extract is present in an amount of about 100 mg to about 800 mg per serving of Composition C. For example, a valerian root extract can be present in an amount of about 100 mg to about 200 mg, about 100 mg to about 300 mg, about 100 mg to about 400 mg, about 100 mg to about 500 mg, about 100 mg to about 600 mg, about 100 mg to about 700 mg, about 200 mg to about 300 mg, about 200 mg to about 400 mg, about 200 mg to about 500 mg, about 200 mg to about 600 mg, about 200 mg to about 700 mg, about 200 mg to about 800, about 300 mg to about 400 mg, about 300 mg to about 500 mg, about 300 mg to about 600 mg, about 300 mg to about 700 mg, about 300 mg to about 800 mg, about 400 mg to about 500 mg, about 400 mg to about 600 mg, about 400 mg to about 700 mg, about 400 mg to about 800 mg, about 500 mg to about 600 mg, about 500 mg to about 700 mg, about 500 mg to about 800 mg, about 600 mg to about 700 mg, about 600 mg to about 800 mg, or about 700 mg to about 800 mg per serving of Composition C. In some embodiments, a valerian root extract is present in an amount of about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, or about 800 mg per serving of Composition C.

In some embodiments, Composition C contains an amount of valerian root extract equivalent to about 1 g to about 9 g of valerian root material per serving (e.g., Composition C can contain an amount of valerian root extract equivalent to about 1 g to about 3 g, 1 g to about 6 g, about 3 g to about 6 g, about 6 g to about 9 g of valerian root powder per serving).

In some embodiments, a valerian root extract can have an extraction ratio of about 9:1, meaning that each milligram of extract is equivalent to 9 milligrams of the plant in its natural state.

Valerian root extracts can contain valerenic acid. In some embodiments, a valerian root extract contains valerenic acid present in an amount of about 0.1% to about 0.8% w/w of the valerian root extract. For example, the valerian root extract can contain valerenic acid present in an amount of about 0.1% to about 0.2%, about 0.1% to about 0.3%, about 0.1% to about 0.4%, about 0.1% to about 0.5%, about 0.1% to about 0.6%, about 0.1% to about 0.7%, about 0.15% to about 0.2%, about 0.15% to about 0.3%, about 0.15% to about 0.4%, about 0.15% to about 0.5%, about 0.15% to about 0.6%, about 0.15% to about 0.7%, about 0.15% to about 0.80%, about 0.2% to about 0.3%, about 0.2% to about 0.4%, about 0.2% to about 0.5%, about 0.2% to about 0.6%, about 0.2% to about 0.7%, about 0.2% to about 0.80%, about 0.25% to about 0.3%, about 0.25% to about 0.4%, about 0.25% to about 0.5%, about 0.25% to about 0.6%, about 0.25% to about 0.7%, about 0.25% to about 0.80%, about 0.3% to about 0.4%, about 0.3% to about 0.5%, about 0.3% to about 0.6%, about 0.3% to about 0.7%, about 0.3% to about 0.80%, about 0.35% to about 0.4%, about 0.35% to about 0.5%, about 0.35% to about 0.6%, about 0.35% to about 0.7%, about 0.35% to about 0.80%, about 0.4% to about 0.45%, about 0.4% to about 0.5%, about 0.4% to about 0.6%, about 0.4% to about 0.7%, about 0.4% to about 0.80%, about 0.45% to about 0.5%, about 0.45% to about 0.6%, about 0.45% to about 0.7%, about 0.45% to about 0.80%, about 0.5% to about 0.6%, about 0.5% to about 0.7%, about 0.5% to about 0.80%, about 0.55% to about 0.6%, about 0.55% to about 0.7%, about 0.55% to about 0.80%, about 0.6% to about 0.7%, about 0.6% to about 0.80%, about 0.65% to about 0.7%, about 0.65% to about 0.80%, or about 0.7% to about 0.80% w/w of the valerian root extract. In some embodiments, the valerian root extract contains valerenic acid present in an amount of about 0.1%, 0.2%, 0.3%, 0.4%, 0.5%, 0.6%, 0.7%, or 0.8% w/w of the valerian root extract.

Combinations of the melatonin, L-theanine, GABA, and ashwagandha of Composition C may have not only a beneficial effect but may also have a synergistic effect. For example, two or more of the melatonin, L-theanine, GABA, and ashwagandha may have an effect that is greater when used together than the sum of the effects that results from using the two or more components separately. See, e.g., Kim et al. Pharm Biol. 57 (1): 65-73 (2019).

In some embodiments, the combination of GABA and L-theanine in Composition C has a synergistic effect. For example, the combination of GABA and L-theanine in Composition C can improve sleep quality and/or sleep duration more than when either GABA or L-theanine is used separately. In some embodiments, GABA and L-theanine are present in Composition C at a ratio of about 5:1 to about 1:3, wherein the ratio is the amount of GABA to the amount of L-theanine (e.g., in mg per serving) in Composition C. In some embodiments, GABA and L-theanine are present in Composition C at a ratio of about 3:2.

In some embodiments, a mood support composition comprising Composition A and Composition C is a composition that includes the ingredients listed in Table 7.

TABLE 7

| | Amount | Amount (% w/w of |
|---|---|---|
| Ingredient | (per serving) | total composition) |
| Lavender | 49 mg to 490 mg | 8% to 16% |
| Lemon Balm | 11 mg to 117 mg | 1% to 4% |
| Chamomile | 9 mg to 98 mg | 1% to 3% |
| Inositol | 9 mg to 98 mg | 1% to 3% |
| GABA | 200 mg to 600 mg | 18% to 34% |
| L-Theanine | 100 mg to 300 mg | 9% to 17% |
| Melatonin | 3 mg to 7 mg | 0.2% to 0.6% |
| Ashwagandha extract | 125 mg to 675 mg | 20% to 25% |

In some embodiments, a mood support composition comprising Composition A and Composition C is a composition that includes the ingredients listed in Table 8.

TABLE 8

| | | | Amount (% |
|---|---|---|---|
| | Amount | | w/w of total |
| Ingredient | (per serving) | | composition) |
| Lavender | about 75 mg to about 175 mg | | 50% to 80% |
| Lemon Balm | about 10 mg to about 80 mg | | 10% to 20% |
| Chamomile | about 20 mg to about 70 mg | | 5% to 25% |
| Inositol | about 20 mg to about 70 mg | | 5% to 25% |
| GABA | 300 | mg | 28.9% |
| L-Theanine | 200 | mg | 19.2% |
| Melatonin | 5 | mg | 0.5% |
| Ashwagandha extract | 125 | mg | 12% |

In some embodiments, the lavender is a lavender extract prepared from flowers of L. angustifolia. In some embodiments, the lemon balm is a lemon balm extract prepared from leaves and stems of M. officinalis. In some embodiments, the chamomile is a chamomile extract prepared from flowers M. chamomilla.

In some embodiments, about 40% w/w of the melatonin is released within a first period of time (e.g., about 1 hour) and about 60% w/w of the melatonin is released over a second period of time (e.g., about 60% w/w of the melatonin is released over about 7 hours following the first period of time).

In some embodiments, the ashwagandha extract contains at least about 5% withanolides. In some embodiments, the ashwagandha extract contains about 5% withanolides.

### Composition D

Provided herein are mood support compositions comprising Composition A and Composition D. Composition D is a composition that contains magnesium and vitamin B6.

Non-limiting examples of magnesium that can be used in Composition D include a magnesium amino acid polysaccharide complex, magnesium citrate, magnesium oxide, magnesium chloride, magnesium lactate, magnesium malate, magnesium taurate, magnesium L-threonate, magnesium sulfate, magnesium glycinate, magnesium orotate, magnesium hydroxide, magnesium bisglycinate, and magnesium gluconate.

In some embodiments, magnesium is present in an amount of about 42 mg to about 630 mg per serving of Composition D. For example, magnesium can be present in an amount of about 42 to about 100 mg, about 42 to about 150 mg, about 42 to about 200 mg, about 42 to about 250 mg, about 42 to about 300 mg, about 42 to about 350 mg, about 42 to about 400 mg, about 42 to about 450 mg, about 42 to about 500 mg, about 42 to about 550 mg, about 42 to about 600 mg, about 100 to about 150 mg, about 100 to about 200 mg, about 100 to about 250 mg, about 100 to about 300 mg, about 100 to about 350 mg, about 100 to about 400 mg, about 100 to about 450 mg, about 100 to about 500 mg, about 100 to about 550 mg, about 100 to about 600 mg, about 100 to about 630 mg, about 150 to about 200 mg, about 150 to about 250 mg, about 150 to about 300 mg, about 150 to about 350 mg, about 150 to about 400 mg, about 150 to about 450 mg, about 150 to about 500 mg, about 150 to about 550 mg, about 150 to about 600 mg, about 150 to about 630 mg, about 200 to about 250 mg, about 200 to about 300 mg, about 200 to about 350 mg, about 200 to about 400 mg, about 200 to about 450 mg, about 200 to about 500 mg, about 200 to about 550 mg, about 200 to about 600 mg, about 200 to about 630 mg, about 250 to about 300 mg, about 250 to about 350 mg, about 250 to about 400 mg, about 250 to about 450 mg, about 250 to about 500 mg, about 250 to about 550 mg, about 250 to about 600 mg, about 250 to about 630 mg, about 300 to about 350 mg, about 300 to about 400 mg, about 300 to about 450 mg, about 300 to about 500 mg, about 300 to about 550 mg, about 300 to about 600 mg, about 300 to about 630 mg, about 350 to about 400 mg, about 350 to about 450 mg, about 350 to about 500 mg, about 350 to about 550 mg, about 350 to about 600 mg, about 350 to about 630 mg, about 400 to about 450 mg, about 400 to about 500 mg, about 400 to about 550 mg, about 400 to about 600 mg, about 400 to about 630 mg, about 450 to about 500 mg, about 450 to about 550 mg, about 450 to about 600 mg, about 450 to about 630 mg, about 500 to about 550 mg, about 500 to about 600 mg, about 500 to about 630 mg, about 550 to about 600 mg, about 550 to about 630 mg, or about 600 to about 630 mg per serving of Composition D. In some embodiments, magnesium is present in an amount of about 200 mg to about 300 mg per serving of Composition D. For example, magnesium can be present in an amount of about 200 mg, about 210 mg, about 220 mg, about 230 mg, about 240 mg, about 250 mg, about 260 mg, about 270 mg, about 280 mg, about 290 mg, or about 300 mg per serving of Composition D.

In some embodiments, magnesium is present in an amount of about 15% to about 65% w/w of the total composition (e.g., Composition A and Composition D combined). For example, magnesium can be present in an amount of about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 25% to about 30%, about 25% to about 35%, about 25% to about 40%, about 25% to about 45%, about 25% to about 50%, about 25% to about 55%, about 25% to about 60%, about 25% to about 65%, about 30% to about 35%, about 30% to about 40%, about 30% to about 45%, about 30% to about 50%, about 30% to about 55%, about 30% to about 60%, about 30% to about 65%, about 35% to about 40%, about 35% to about 45%, about 35% to about 50%, about 35% to about 55%, about 35% to about 60%, about 35% to about 65%, about 40% to about 45%, about 40% to about 50%, about 40% to about 55%, about 40% to about 60%, about 40% to about 65%, about 50% to about 55%, about 50% to about 60%, about 50% to about 65%, about 55% to about 60%, about 55% to about 65%, or about 60% to about 65% w/w of the total composition. In some embodiments, magnesium is present in an amount of about 25% to about 40% w/w of the total composition. In some embodiments, magnesium is present in an amount of about 10%, about 15%, about 20%, about 25%, about 30%, about 34%, about 35%, about 36%, about 40%, about 45%, about 50%, about 60%, or about 65% w/w of the total composition.

In some embodiments, the magnesium comprises a magnesium amino acid polysaccharide complex, magnesium citrate, magnesium oxide, magnesium chloride, magnesium lactate, magnesium malate, magnesium taurate, magnesium L-threonate, magnesium sulfate, magnesium glycinate, magnesium orotate, magnesium hydroxide, magnesium bisglycinate, magnesium gluconate, or mixtures thereof. In some embodiments, the magnesium amino acid polysaccharide complex comprises 75% aspartate and 25% glycinate. In some embodiments, the magnesium amino acid polysaccharide complex comprises polyfructose. In some embodiments, the magnesium comprises a magnesium amino acid polyfructose complex.

Vitamin B6 can be present in Composition D as pyridoxine, pyridoxal, and pyridoxamine, pyridoxine 5′-phosphate, pyridoxal 5′-phosphate and pyridoxamine 5′-phosphate.

In some embodiments, vitamin B6 is present in an amount of about 0.15 mg to about 100 mg per serving of Composition D. For example, vitamin B6 can be present in an amount of about 0.15 mg to about 1 mg, about 0.15 mg to about 10 mg, about 0.15 mg to about 25 mg, about 0.15 mg to about 50 mg, about 0.15 mg to about 75 mg, about 1 mg to about 10 mg, about 1 mg to about 25 mg, about 1 mg to about 50 mg, about 1 mg to about 75 mg, about 1 mg to about 100 mg, about 10 mg to about 25 mg, about 10 mg to about 50 mg, about 10 mg to about 75 mg, about 10 mg to about 100 mg, about 25 mg to about 50 mg, about 25 mg to about 75 mg, about 25 mg to about 100 mg, about 50 mg to about 75 mg, about 50 mg to about 100 mg, or about 75 mg to about 100 mg per serving of Composition D. In some embodiments, vitamin B6 is present in an amount of about 25 mg to about 60 mg per serving of Composition D. For example, vitamin B6 can be present in an amount of about 25 mg, about 30 mg, about 35 mg, about 40 mg, about 45 mg, about 50 mg, about 55 mg, or about 60 mg per serving of Composition D.

In some embodiments, vitamin B6 is present in an amount of about 1% to about 10% w/w of the total composition (e.g., Composition A and Composition D combined). For example, vitamin B6 can be present in an amount of about 1% to about 2%, about 1% to about 4%, about 1% to about 6%, about 1% to about 8%, about 2% to about 4%, about 2% to about 6%, about 2% to about 8%, about 2% to about 10%, about 4% to about 6%, about 4% to about 8%, about 4% to about 10%, about 6% to about 8%, about 6% to about 10%, or about 8% to about 10% w/w of the total composition. In some embodiments, vitamin B6 is present in an amount of about 2% to about 5% w/w of the total composition. In some embodiments, vitamin B6 is present in an amount of about 1%, about 2%, about 3%, about 4%, about 5%, about 6%, about 7%, about 8%, about 9%, or about 10% w/w of the total composition.

In some embodiments, a mood support composition provided herein can be a composition that includes ingredients as shown in Table 9.

TABLE 9

| | Amount | Amount |
|---|---|---|
| Ingredient | (per serving) | (% w/w of total) |
| Lavender | 49 mg to 490 mg | 3% to 20% |
| Lemon Balm | 11 mg to 117 mg | 2% to 5% |
| Chamomile | 9 mg to 98 mg | 2% to 5% |
| Inositol | 9 mg to 98 mg | 2% to 5% |
| Magnesium | 200 mg to 300 mg | 24% to 40% |
| Vitamin B6 | 25 mg to 60 mg | 2% to 5% |

In some embodiments, a mood support composition provided herein can be a composition that includes ingredients as shown in Table 10.

TABLE 10

| | Amount |
|---|---|
| Ingredient | (per serving) |
| Lavender | about 75 mg to about 175 mg |
| Lemon Balm | about 10 mg to about 80 mg |
| Chamomile | about 20 mg to about 70 mg |
| Inositol | about 20 mg to about 70 mg |
| Magnesium | 300 mg |
| Vitamin B6 | 200 mg |

### Formulations

A mood support composition provided herein (e.g., Composition A or a composition comprising Composition A and one or more of Composition B and Composition C) can be formulated for oral use and can further include one or more suitable excipients, sweetening agents, flavorants, colorants, preservatives, and/or other ingredients. Non-limiting examples of oral formulations include tablets, troches, lozenges, aqueous or oil suspensions, non-aqueous solutions, dispersible powders or granules (including micronized particles or nanoparticles), emulsions, hard or soft capsules, syrups, and elixirs. Compositions intended for oral use can be prepared according to any appropriate method, including standard methods for the manufacture of pharmaceutical compositions. Preparations for oral use can be suitably formulated to give controlled release of one or more compounds.

Liquid preparations for oral administration can take the form of, for example, solutions, syrups, or suspension, or they can be presented as a dry product for reconstitution with saline or other suitable liquid vehicle before use. In some embodiments, liquid preparations can contain pharmaceutically acceptable additives such as suspending agents, emulsifying agents, non-aqueous vehicles, preservatives, buffer salts, flavoring agents, coloring agents, and sweetening agents as appropriate.

Tablets, capsules, or soft gels can be prepared with one or more suitable excipients, such as binding agents, fillers, lubricants, disintegrants, and/or wetting agents. In some embodiments, a capsule, tablet, or soft gel form of the mood support compositions provided herein further includes one or more of microcrystalline cellulose, cellulose gum, inulin, calcium sulfate, carnauba wax, glycerin, hydroxypropyl cellulose, hydroxypropyl methylcellulose, isomalt, magnesium stearate, rice starch, silicon dioxide, and stearic acid. In some embodiments, a capsule, tablet, or soft gel form of the mood support compositions provided herein further includes one or more of microcrystalline cellulose, an amorphous silica, magnesium stearate, and hydroxypropyl methylcellulose. In some embodiments, a capsule, tablet, or soft gel form of the mood support compositions provided herein further includes one or more of microcrystalline cellulose, magnesium stearate, and hydroxypropyl methylcellulose.

In some embodiments, a tablet of a mood support composition provided herein can have a coating (e.g., a polymer or polysaccharide-based coating with or without plasticizers and/or pigments). Such coatings can include, for example, one or more of a film-forming polymer, a colorant, and a flavorant. Non-limiting examples of a flavorant include vanillin, vanilla extract, artificial vanilla flavorant, and natural caramel. In some embodiments, a mood support composition can have a vanilla or vanilla caramel flavor profile. In some embodiments, a coating of a tablet of a mood support composition provided herein includes one or more of: hydroxypropyl methyl cellulose, sodium alginate, calcium carbonate, calcium sulfate, magnesium carbonate, cellulose gum, isomalt, polyethylene glycol, glycerin, hydroxypropyl cellulose, and carnauba wax.

In some embodiments, a capsule, tablet, or soft gel form of the mood support compositions provided herein can have a unit dosage equal to the daily desired dosage for a particular mammal. For example, if it is desired to administer 100 mg of a particular agent to a mammal, each tablet can include about 100 mg in weight of that agent. As used herein, mammals generally refer to humans, but also can include domesticated mammals (e.g., dogs, cats, and livestock such as cows, horses, pigs, or sheep). The dosages of a particular mood support composition provided herein will depend on many factors, including the general health of a mammal. In some cases, a total daily dose (e.g., a full serving) can be prepared and administered in the form of one or more dosage units (e.g., one tablet, capsule, or soft gel, two tablets, capsules, or soft gels, three tablets, capsules, or soft gels, four tablets, capsules or soft gels, five tablets, capsules, or soft gels, or six tablets, capsules, or soft gels). For instance, in some cases, one tablet, one capsule, or one soft gel can include a full serving (e.g., daily dosage) of the composition. In some cases, two tablets, two capsules, or two soft gels in combination can include a full serving (e.g., daily dosage) of the composition. In some cases, three tablets, three capsules, or three soft gels in combination can include a full serving (e.g., daily dosage) of the composition.

In some embodiments, two capsules in combination contain a full serving (e.g., daily dosage) of a mood support composition provided herein.

In some embodiments, a mood support composition provided herein can be substantially free of artificial colors, artificial flavors, and chemical preservatives.

In some embodiments, a mood support composition provided herein can be a composition that includes ingredients as shown in Table 11.

TABLE 11

| | Amount | | Amount (% w/w of |
|---|---|---|---|
| Ingredient | (per serving) | | total composition) |
| Lavender | 75 mg to 175 mg | | 3% to 60% |
| Lemon Balm | 10 mg to 80 mg | | 0.75% to 15% |
| Chamomile | 20 mg to 70 mg | | 0.6% to 11% |
| Inositol | 20 mg to 70 mg | | 5% to 25% |
| Saffron extract | 28 | mg | 0.25% to 5% |
| Ashwagandha extract | 600 | mg | 5% to 80% |
| Vitamin B12 | 6 | μg | 0.00001% to 0.001% |
| Folate | 400 | μg | 0.0001% to 0.1% |

| | |
|---|---|
| Binding agent(s), filler(s), | 10% to 90% |
| lubricant(s), disintegrant(s), | |
| colorant(s), and flavorant(s) | |

In some embodiments, a mood support composition provided herein can be a composition that includes ingredients as shown in Table 12.

TABLE 12

| | Amount | | Amount (% w/w of |
|---|---|---|---|
| Ingredient | (per serving) | | total composition) |
| Lavender | 75 mg to 175 mg | | 3% to 60% |
| Lemon Balm | 10 mg to 80 mg | | 0.75% to 15% |
| Chamomile | 20 mg to 70 mg | | 0.6% to 11% |
| Inositol | 20 mg to 70 mg | | 5% to 25% |
| GABA | 300 | mg | 28.9% |
| L-Theanine | 200 | mg | 19.2% |
| Melatonin | 5 | mg | 0.5% |
| Ashwagandha extract | 125 | mg | 12% |

| | |
|---|---|
| Binding agent(s), filler(s), | |
| lubricant(s), disintegrant(s), | 10% to 90% |
| colorant(s), and flavorant(s) | |

In some embodiments, a mood support composition provided herein can be a composition that includes ingredients as shown in Table 13.

TABLE 13

| | Amount | Amount (% w/w of |
|---|---|---|
| Ingredient | (per serving) | total composition) |
| Lavender | 75 mg to 175 mg | 3% to 60% |
| Lemon Balm | 10 mg to 80 mg | 0.75% to 15% |
| Chamomile | 20 mg to 70 mg | 0.6% to 11% |
| Inositol | 20 mg to 70 mg | 5% to 25% |
| Magnesium | 300 mg | 25% to 40% |
| Vitamin B6 | 200 mg | 2% to 5% |
| Binding agent(s), filler(s), | | 10% to 90% |
| lubricant(s), disintegrant(s), | | |
| colorant(s), and flavorant(s) | | |

### Methods

The mood support compositions provided herein can be useful for improving one or more aspects related to mood, e.g., one or more of supporting a positive mental outlook, better coping with the ups and downs of life, maintaining a balanced mood, promoting emotional health, supporting emotional balance, supporting the activities of brain messengers, supporting general well-being, supporting more restful sleep (e.g., restful sleep without sedation), reducing feelings of increased sadness, reducing feelings of occasional stress, supporting mood-related sexual health, and promoting physical and mental energy. For example, a mammal may feel less stressed, feel more positive, and/or feel more energetic after consuming a mood support composition provided herein as compared to a day where a mood support composition provided herein was not consumed.

Provided herein are methods of improving one or more aspects related to mood in a mammal, comprising administering to the mammal any of the compositions described herein. In some embodiments, improving an aspect related to mood comprises one or more of improving emotional health, improving the ability to cope with stress, improving the activity of a brain messenger, improving emotional balance, improving general well-being, improving the restfulness of sleep, and improving physical and mental energy.

Also provided herein are methods of reducing feelings of sadness and/or feelings of stress in a mammal, comprising administering to the mammal any of the compositions described herein.

One or more aspects related to mood can each be assessed using any appropriate method(s). Non-limiting examples of methods that can be used to assess one or more of supporting a positive mental outlook, better coping with the ups and downs of life, maintaining a balanced mood, promoting emotional health, supporting emotional balance, supporting the activities of brain messengers, supporting general well-being, supporting more restful sleep (e.g., restful sleep without sedation), reducing feelings of increased sadness, reducing feelings of occasional stress, supporting mood-related sexual health, and promoting physical and mental energy include Profile of Mood States (POMS) survey, HAM-D, HAM-A, Mental Fatigue, Alertness, and Motivation Visual Analogue Scales (VAS), Mental and Physical State Energy and Fatigue Scales. See, e.g., Reed et al. J. Cogn. Enhanc. 3:193-206 (2019); Tartar et al. Cureus 13 (2021).

POMS is a self-report questionnaire that is widely used to measure mood. See e.g., Morfeld et al. Psychosoc. Med. 4: Doc06 (2007). HAM-D and HAM-A are clinician-administered depression assessment and anxiety assessment scales, respectively. See, e.g., Rohan, et al. J Affect Disord. 200:111-118 (2016); and Lopresti et al. Medicine (Baltimore). 98 (37): e17186 (2019). Mental Fatigue, Alertness, and Motivation Visual Analogue Scales can be used to assess current feelings of mental fatigue, alertness, and motivation. For example, the mental fatigue and alertness scales can be scored from “not at all” to “extremely,” while the motivation scale can be scored from “no motivation at all” to “strongest degree of motivation imaginable,” with scores ranging from 0 to 100. Mental and Physical State Energy and Fatigue Scales (EFS-State Scales) can include reporting of a mammal's (e.g., a human) current subjective feelings of physical energy, physical fatigue, mental energy, and mental fatigue.

In some embodiments, a mood support composition provided herein inhibits glycogen synthase kinase-3 (GSK3). In some embodiments, a mood support composition provided herein inhibits GSK3α and/or GSK3β.

In some embodiments, the combination of lavender, lemon balm, chamomile, and inositol has a synergistic effect. For example, the combination of lavender, lemon balm, chamomile, and inositol can improve one or more aspects related to mood more than when lavender, lemon balm, chamomile, or inositol are used separately. In some embodiments, the combination of lavender, lemon balm, chamomile, and inositol can improve one or more aspects related to mood more than the additive effect expected from lavender, lemon balm, chamomile, or inositol when used separately.

In some embodiments, an improvement in an aspect related to mood occurs within about 1 day to about 8 weeks of consuming a mood composition as provided herein. For example, within about 1 day to about 1 week, about 1 day to about 2 weeks, about 1 day to about 3 weeks, about 1 day to about 4 weeks, about 1 day to about 6 weeks, about 1 week to about 2 weeks, about 1 week to about 3 weeks, about 1 week to about 4 weeks, about 1 week to about 6 weeks, about 1 week to about 8 weeks, about 2 weeks to about 3 weeks, about 2 weeks to about 4 weeks, about 2 weeks to about 6 weeks, about 2 weeks to about 8 weeks, about 3 weeks to about 4 weeks, about 3 weeks to about 6 weeks, about 3 weeks to about 8 weeks, about 4 weeks to about 6 weeks, about 4 weeks to about 8 weeks, or about 6 weeks to about 8 weeks of consuming a mood composition as provided herein. In some embodiments, an improvement in an aspect related to mood occurs within about 2 weeks to about 4 weeks of consuming a mood composition as provided herein.

The frequency of consuming a mood support composition provided herein can vary from once or twice a week to once or more times a day, such as two or three times daily. In some embodiments, a mood support composition provided herein is consumed once daily. The frequency of consuming a mood support composition provided herein can be constant or be variable during the duration of the treatment.

In some embodiments, wherein a total daily dose (e.g., a full serving) of a mood support composition provided herein is consumed in the form of two or more dosage units, at least one of the dosage units is consumed at a different time than at least one other dosage unit. For example, when two tablets, capsules, or soft gels together contain a full serving of a mood support composition provided herein, one capsule is consumed in the morning and another capsule is consumed in the evening.

### EXAMPLES

### Example 1. Exemplary Mood Support Composition

A mood support composition was produced having the ingredients shown in Table 14.

TABLE 14

| | Amount | |
|---|---|---|
| Ingredient | (per serving) | Amount (% w/w) |
| Lavender | about 75 mg to about 175 mg | about 55% to about 65% |
| Lemon Balm | about 10 mg to about 80 mg | about 10% to about 20% |
| Chamomile | about 20 mg to about 70 mg | about 10% to about 15% |
| Inositol | about 20 mg to about 70 mg | about 10% to about 15% |
| Total | 205 mg | |

A mood support composition was produced having the ingredients shown in Table 15.

TABLE 15

| | | | Amount (% |
|---|---|---|---|
| | Amount | | w/w of total |
| Ingredient | (per serving) | | composition) |
| Lavender | about 75 mg to about 175 mg | | 10% to 15% |
| Lemon Balm | about 10 mg to about 80 mg | | 1% to 5% |
| Chamomile | about 20 mg to about 70 mg | | 1% to 5% |
| Inositol | about 20 mg to about 70 mg | | 1% to 5% |
| GABA | 300 | mg | 28.9% |
| L-Theanine | 200 | mg | 19.2% |
| Melatonin | 5 | mg | 0.5% |
| Ashwagandha extract | 125 | mg | 12% |

| | |
|---|---|
| Binding agent(s), filler(s), | 10% to 27% |
| lubricant(s), disintegrant(s), | |
| colorant(s), and flavorant(s) | |

A mood support composition is produced having the ingredients shown in Table 16.

TABLE 16

| | Amount | | Amount (% w/w of |
|---|---|---|---|
| Ingredient | (per serving) | | total composition) |
| Lavender | 75 mg to 175 mg | | 3% to 60% |
| Lemon Balm | 10 mg to 80 mg | | 0.75% to 15% |
| Chamomile | 20 mg to 70 mg | | 0.6% to 11% |
| Inositol | 20 mg to 70 mg | | 5% to 25% |
| Ashwagandha extract | 600 | mg | 30% to 60% |
| Vitamin B12 | 6 | μg | 0.00001% to 0.01% |
| Folate | 400 | μg | 0.001% to 0.1% |

| | |
|---|---|
| Binding agent(s), filler(s), | 10% to 90% |
| lubricant(s), disintegrant(s), | |
| colorant(s), and flavorant(s) | |

A mood support composition is produced having the ingredients shown in Table 16 and further including 28 mg of a saffron extract (e.g., about 1% to about 8% w/w of the total composition).

### Example 2. Synergistic Effects of a Mood Support Composition

The cellular mechanism of action of known pharmaceutical compounds targeting mental health require the inhibition of individual enzymes. For example, one of the most well-known pharmaceuticals utilized in the mental health space, lithium, can trace its efficacy in part to the inhibition of an enzyme called glycogen synthase kinase 3 (GSK3). A technique similar to one used in the pharmaceutical industry was used to determine study a mood support composition. See, e.g., Duong-Ly, K. C. & Peterson, J. R. Curr. Protoc. Pharmacol. Chapter 2, Unit2.9 (2013); Ma, H. et al. Expert Opin. Drug Discov. 3, 607-621 (2008).

Within the body, protein kinases are responsible for carrying out cellular and intracellular actions, such as cell-to-cell signaling, neurotransmitter regulation, and hormone release. Several protein kinases are known to influence mood, and many are targets of therapeutic drugs. As a natural alternative to pharmaceuticals, Blend A was screened for efficacy of inhibiting 18 common, mood-related drug targets, including 17 kinases and 1 enzyme.

### Methods

Blends and samples were prepared on the bench in stock samples of ACS grade DMSO to a target concentration of 5 mg/mL. The one exception was the inositol ingredient which had limited solubility in DMSO, but it was soluble in water. These concentrated stocks were then serially diluted to appropriate concentrations, with the minimum dilution being 50:1. Blend A was prepared as 61% lavender, 15% lemon balm, 12% chamomile and 12% inositol.

Per the literature the expected IC50 was calculated using the proportional inverse sum of the components, as shown in the equation:

$$\frac{1}{IC {50}_{expected}} = \frac{{F}_{A}}{IC {50}_{A}} + \frac{{F}_{B}}{IC {50}_{B}} + … \frac{{F}_{n}}{IC {50}_{n}}$$

See Babish, J. G. et al. Can. J. Physiol. Pharmacol. 94 (12): 1257-1266 (2016).

One modification that was made from the literature was to normalize to the standard used for the assays. In other words, the IC50 was presented as IC50ₙ/IC50_{Staurosporine}. This was done because Blend A was prepared and assayed several times, several months apart (first screening, then with the components). Thus, the relative IC50 was used in the equation for each of the components to calculate the expected, and the relative IC50 of each of the analyses of Blend A was used to calculate the blend's IC50—each test carrying equal (50%) weight in the equation.

The results of the initial investigation indicated the strongest effects were present on GRK2, GSK3a, and GSK3b, which are targets of proven, mood-modulating drugs, Paroxetine (GRK2) and Lithium (GSK3). A follow up investigation was then performed on the individual ingredients in addition to Blend A, which was reprepared.

### Results

The results of the initial investigation indicated the strongest effects were present on GRK2, GSK3α, and GSK3β, which are targets of proven, mood-modulating drugs, Paroxetine (GRK2) and Lithium (GSK3). The control compounds were SCH772984, an ERK inhibitor, or staurosporine. See Table 17 below.

TABLE 17

| | Blend A IC50* | Control | |
|---|---|---|---|
| Kinase: | (M) | IC50 (M) | Control Compound |
| ERK1 | >1.00E−03 | 5.57E−09 | SCH772984 |
| ERK2/MAPK1 | 6.36E−04 | 6.37E−10 | SCH772984 |
| GRK2 | 7.86E−05 | 8.33E−07 | STAUROSPORINE |
| GSK3α | 5.85E−06 | 2.49E−09 | STAUROSPORINE |
| GSK3β | 2.66E−06 | 7.66E−09 | STAUROSPORINE |
| MEK1 | 5.51E−04 | 4.11E−08 | STAUROSPORINE |
| MEK2 | 3.87E−04 | 5.26E−08 | STAUROSPORINE |
| MEK3 | >1.00E−03 | 1.42E−08 | STAUROSPORINE |
| PKA | 1.33E−04 | 1.83E−09 | STAUROSPORINE |
| PKAcb | 5.86E−04 | 1.85E−09 | STAUROSPORINE |
| PKAcg | 3.29E−04 | 8.48E−09 | STAUROSPORINE |
| PKCa | 2.76E−04 | 2.07E−10 | STAUROSPORINE |
| PKCb1 | 1.13E−03 | 7.69E−10 | STAUROSPORINE |
| PKCb2 | 1.26E−04 | 1.38E−10 | STAUROSPORINE |
| PKCepsilon | 6.20E−04 | 2.15E−10 | STAUROSPORINE |
| PKCg | 3.04E−04 | 1.60E−10 | STAUROSPORINE |
| PKCIOTA | 1.38E−04 | 1.41E−08 | STAUROSPORINE |
| PKCtheta | 2.12E−04 | 3.63E−10 | STAUROSPORINE |

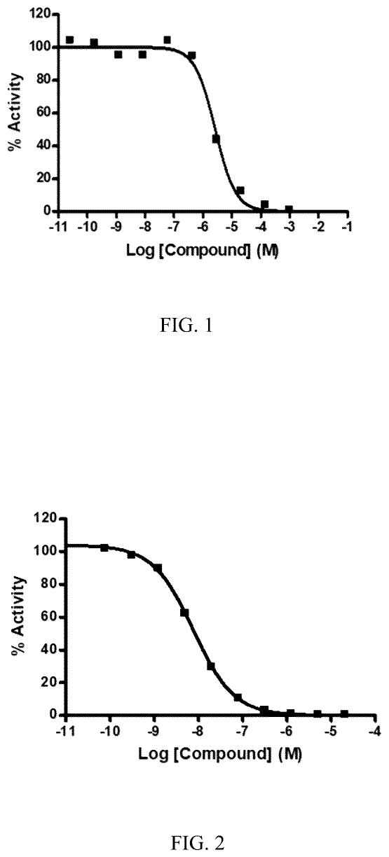

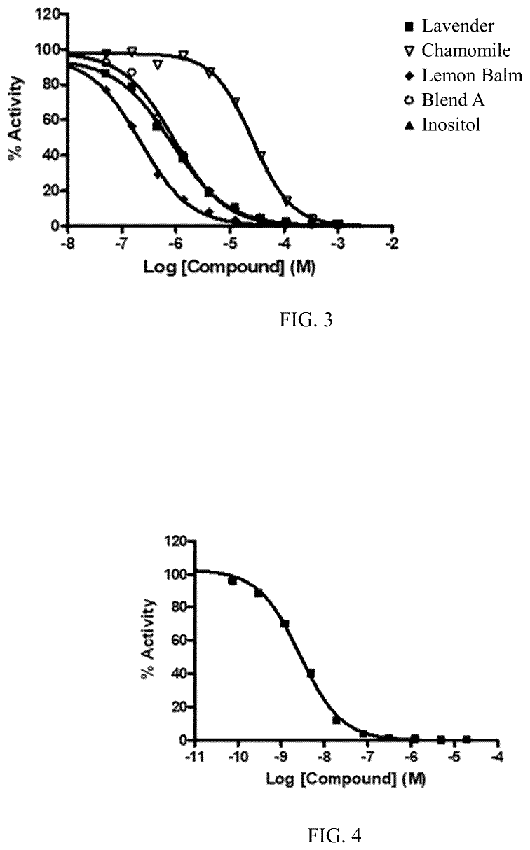

A follow up investigation was then performed on the individual ingredients in addition to Blend A, which was reprepared. Results are shown in Table 18 below. See also FIGS. 1-4. The control compound was staurosporine.

TABLE 18

| | Compound IC50* (M): | | | | | IC50 (M) |
|---|---|---|---|---|---|---|
| Kinase: | Lavender | Chamomile | Lemon Balm | Blend A | Inositol | Control |

| | | | | | |
|---|---|---|---|---|---|
| GRK2 | 7.30E−05 | >1.00E−03 | 8.34E−06 | 5.40E−05 | 4.99E−07 |
| GSK3a | 8.29E−07 | 2.61E−05 | 2.19E−07 | 9.15E−07 | 2.70E−09 |
| GSK3b | 5.53E−06 | 3.90E−05 | 2.17E−06 | 7.98E−06 | 4.30E−09 |

The most consistently active individual ingredient was the lemon balm against the 3 assays. Inositol did not demonstrate efficacy independently for inhibiting any of the 3 targets, so the upper limit of use (1 mM) was used. In some cases, the individual ingredients had better potency than the blend reports, however as the blend dilutes out the potential of that activity, the expected IC50 for the blend becomes less potent. To put this another way, the most active ingredient (lemon balm) is only used at 15% by weight in the blend so the expected activity contribution is 38% overall, while the lavender represents 61% of the blend's weight and contributes about 60% to the overall activity—this means the remaining 24% of weight contributes <2% to the overall expected activity.

To best represent the sample in a practical setting (such as full-scale production), data were normalized to the staurosporine standard of their respective study and aggregated with each of the 2 standards and each Blend A contributing 50% weight. Results are shown in Table 19 below.

TABLE 19

| | | Contributed IC50 | | |
|---|---|---|---|---|
| | wt % | GRK2 | GSK3a | GSK3b |
| Lavender | 61% | 0.0042 | 0.002 | 0.00047 |
| Lemon Balm | 15% | 0.009 | 0.0018 | 0.0003 |
| Chamomile | 12% | 1.00E−05 | 1.24E−05 | 1.32E−05 |
| Inositol | 12% | 0.00006 | 3.24E−07 | 5.16E−07 |
| Blend A 1 | 50% | 0.00538 | 0.00021 | 0.0014 |
| Blend A 2 | 50% | 0.0046 | 0.0015 | 0.00027 |
| Staurosporine 1 | 50% | 6.00E+05 | 2.01E+08 | 6.53E+07 |
| Staurosporine 2 | 50% | 1.00E+06 | 1.85E+08 | 1.16E+08 |
| Ingredient Reciprocal | | 0.0132 | 0.0038 | 0.00079 |
| Sum | | | | |
| Blend Reciprocal Sum | | 0.001 | 0.0017 | 0.0017 |
| Staurosporine Reciprocal | | 1.60E+06 | 3.86E+08 | 1.82E+08 |
| Sum | | | | |
| IC50/Ing | | 76 | 260 | 1273 |
| IC50/Blend | | 101 | 593 | 585 |
| Avg Staurosp | | 6.2E-07 | 2.6E-09 | 5.5E-09 |
| IC50 ing | | 4.7E-05 | 6.7E-07 | 7.0E-06 |
| IC50 Blend | | 6.3E-05 | 1.5E-06 | 3.2E-06 |
| | | 0.75 | 0.44 | 2.18 |

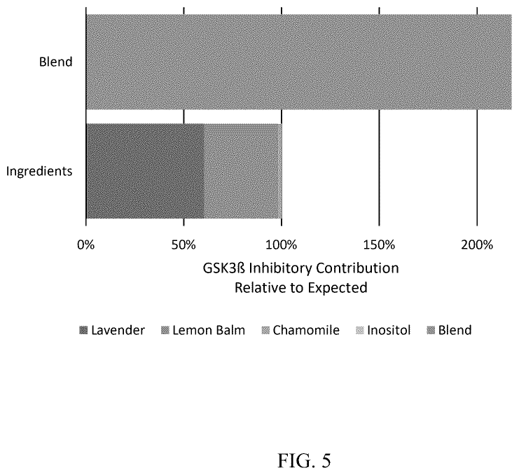

For GSK3β, Blend A was found to be 218% better at inhibition (lower IC50) than was expected from the sum of the parts. See also FIG. 5.

### Exemplary Embodiments

An embodiment described herein provides a mood support composition. The mood support composition includes a lavender extract, a lemon balm extract, a chamomile extract, and inositol.

In an embodiment, combinable with any other embodiment, the lavender extract is present in an amount of about 12 mg to about 1220 mg per serving of the mood support extract.

In an embodiment, combinable with any other embodiment, the lavender extract is present in an amount of about 49 mg to about 490 mg per serving of the mood support extract.

In an embodiment, combinable with any other embodiment, the lemon balm extract is present in an amount of about 2 mg to about 293 mg per serving of the mood support extract.

In an embodiment, combinable with any other embodiment, the lemon balm extract is present in an amount of about 11 mg to about 117 mg per serving of the mood support extract.

In an embodiment, combinable with any other embodiment, the chamomile extract is present in an amount of about 2 mg to about 244 mg per serving of the mood support extract.

In an embodiment, combinable with any other embodiment, the chamomile extract is present in an amount of about 9 mg to about 98 mg per serving of the mood support extract.

In an embodiment, combinable with any other embodiment, the inositol is present in an amount of about 2 mg to about 244 mg per serving of the mood support extract.

In an embodiment, combinable with any other embodiment, the inositol is present in an amount of about 9 mg to about 98 mg per serving of the mood support extract.

In an embodiment, combinable with any other embodiment, the mood support composition further includes one or more of: an ashwagandha extract, a folate, vitamin B12, where the vitamin B12 includes methylcobalamin.

In an embodiment, combinable with any other embodiment, the mood support composition further includes one or more of: a saffron extract, an ashwagandha extract, a folate, vitamin B12, where the vitamin B12 includes methylcobalamin.

In an embodiment, combinable with any other embodiment, the saffron extract is present in an amount of about 14 mg to about 56 mg per serving of the mood support composition.

In an embodiment, combinable with any other embodiment, the saffron extract is present in an amount of about 28 mg to about 30 mg per serving of the mood support composition.

In an embodiment, combinable with any other embodiment, the saffron extract is present in an amount of about 28 mg per serving of the mood support composition.

In an embodiment, combinable with any other embodiment, the saffron extract is an extract of the stigmas of Crocus sativus flowers.

In an embodiment, combinable with any other embodiment, the ashwagandha extract is present in an amount of about 125 mg to about 1800 mg per serving of the mood support composition.

In an embodiment, combinable with any other embodiment, the ashwagandha extract is present in an amount of about 300 mg to about 675 mg per serving of the mood support composition.

In an embodiment, combinable with any other embodiment, the ashwagandha extract is present in an amount of about 600 mg per serving of the mood support composition.

In an embodiment, combinable with any other embodiment, the ashwagandha extract contains one or more withanolides.

In an embodiment, combinable with any other embodiment, the ashwagandha extract is an ashwagandha root extract.

In an embodiment, combinable with any other embodiment, vitamin B12 is present in an amount of about 0.5 μg to about 100 μg per serving of the mood support composition.

In an embodiment, combinable with any other embodiment, vitamin B12 is present in an amount of about 3 μg to about 60 μg per serving of the mood support composition.

In an embodiment, combinable with any other embodiment, vitamin B12 is present in an amount of about 6 μg per serving of the mood support composition.

In an embodiment, combinable with any other embodiment, the folate is present in an amount of about 0.1 mg to about 2 mg DFE per serving of the mood support composition. In an embodiment, combinable with any other embodiment, the folate is present in an amount of about 0.2 mg to about 0.8 mg DFE per serving of the mood support composition.

In an embodiment, combinable with any other embodiment, the folate is present in an amount of about 0.4 mg DFE per serving of the mood support composition.

In an embodiment, combinable with any other embodiment, the folate comprises calcium methyltetrahydrofolate.

In an embodiment, combinable with any other embodiment, the mood support composition further including melatonin, L-theanine, GABA (gamma-aminobutyric acid), and an ashwagandha extract.

In an embodiment, combinable with any other embodiment, the melatonin is present in an amount of about 1 mg to about 10 mg per serving.

In an embodiment, combinable with any other embodiment, the melatonin is present in an amount of about 3 mg to about 7 mg per serving.

In an embodiment, combinable with any other embodiment, the melatonin is present in an amount of about 5 mg per serving.

In an embodiment, combinable with any other embodiment, the mood support composition is formulated for rapid release and sustained release of the melatonin.

In an embodiment, combinable with any other embodiment, about 20% to about 60% w/w of the melatonin is released within a first period of time.

In an embodiment, combinable with any other embodiment, about 40% w/w of the melatonin is released within a first period of time.

In an embodiment, combinable with any other embodiment, the first period of time is about 30 minutes to about 1 hour.

In an embodiment, combinable with any other embodiment, the first period of time is about 1 hour.

In an embodiment, combinable with any other embodiment, about 40% to about 80% w/w of the melatonin is released over a second period of time following the first period of time.

In an embodiment, combinable with any other embodiment, about 60% w/w of the melatonin is released within the second period of time.

In an embodiment, combinable with any other embodiment, the first period of time is about 6 hours to about 8 hours.

In an embodiment, combinable with any other embodiment, the second period of time is about 7 hours.

In an embodiment, combinable with any other embodiment, the L-theanine is present in an amount of about 25 mg to about 500 mg per serving.

In an embodiment, combinable with any other embodiment, the L-theanine is present in an amount of about 100 mg to about 300 mg per serving.

In an embodiment, combinable with any other embodiment, the L-theanine is present in an amount of about 200 mg per serving.

In an embodiment, combinable with any other embodiment, the GABA is present in an amount of about 100 mg to about 1000 mg per serving.

In an embodiment, combinable with any other embodiment, the GABA is present in an amount of about 200 mg to about 600 mg per serving.

In an embodiment, combinable with any other embodiment, the GABA is present in an amount of about 300 mg per serving.

In an embodiment, combinable with any other embodiment, the ashwagandha extract is present in an amount of about 50 mg to about 1800 mg per serving.

In an embodiment, combinable with any other embodiment, the ashwagandha extract is present in an amount of about 125 mg to about 675 mg per serving.

In an embodiment, combinable with any other embodiment, the ashwagandha extract is present in an amount of about 125 mg per serving.

In an embodiment, combinable with any other embodiment, the ashwagandha extract contains one or more withanolides.

In an embodiment, combinable with any other embodiment, the one or more withanolides are present in an amount of about 1% to about 12% w/w of the ashwagandha extract.

In an embodiment, combinable with any other embodiment, the one or more withanolides are present in an amount of about 5% w/w of the ashwagandha extract.

In an embodiment, combinable with any other embodiment, the mood support composition further includes a valerian root extract.

In an embodiment, combinable with any other embodiment, the lavender and lemon balm are present at a ratio of about 25:1 to about 1:1 lavender:lemon balm.

In an embodiment, combinable with any other embodiment, the lavender and lemon balm are present at a ratio of about 5:1 to about 1:1 lavender:lemon balm.

In an embodiment, combinable with any other embodiment, the lavender and lemon balm are present at a ratio of about 4:1 lavender:lemon balm.

In an embodiment, combinable with any other embodiment, the chamomile and inositol are present at a ratio of about 25:1 to about 1:25 chamomile:inositol.

In an embodiment, combinable with any other embodiment, the chamomile and inositol are present at a ratio of about 2:1 to about 1:2 chamomile:inositol.

In an embodiment, combinable with any other embodiment, the chamomile and inositol are present at a ratio of about 1:1 chamomile:inositol.

In an embodiment, combinable with any other embodiment, the lavender and lemon balm and the chamomile and inositol are present at a ratio of about 25:1 to about 1:1 lavender and lemon balm: chamomile and inositol.

In an embodiment, combinable with any other embodiment, the lavender and lemon balm and the chamomile and inositol are present at a ratio of about 4:1 to about 1:1 lavender and lemon balm: chamomile and inositol.

In an embodiment, combinable with any other embodiment, the lavender and lemon balm and the chamomile and inositol are present at a ratio of about 3:1 lavender and lemon balm: chamomile and inositol.

In an embodiment, combinable with any other embodiment, the mood support composition is formulated as a tablet, a capsule, or a soft gel.

In an embodiment, combinable with any other embodiment, two tablets, capsules, or soft gels comprises a full serving of the mood support composition.

In an embodiment, combinable with any other embodiment, the mood support composition is formulated as a capsule.

An embodiment provided herein is a method of improving one or more aspects related to mood in a mammal, the method including administering to the mammal the mood support composition of any other embodiment.

In an embodiment, combinable with any other embodiments, improving an aspect related to mood includes one or more of improving emotional health, improving the ability to cope with stress, improving the activity of a brain messenger, improving emotional balance, improving general well-being, improving the restfulness of sleep, and improving physical and mental energy.

An embodiment provided herein is a method of reducing feelings of sadness and/or feelings of stress in a mammal, the method including administering to the mammal the mood support composition of any other embodiment.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A mood support composition comprising:
a lavender extract;
a lemon balm extract;
a chamomile extract; and
inositol.

2. The mood support composition of claim 1, wherein the lavender extract is present in an amount of about 12 mg to about 1220 mg per serving of the mood support extract.

3. The mood support composition of claim 1, wherein the lemon balm extract is present in an amount of about 2 mg to about 293 mg per serving of the mood support extract.

4. The mood support composition of claim 1, wherein the chamomile extract is present in an amount of about 2 mg to about 244 mg per serving of the mood support extract.

5. The mood support composition claim 1, wherein the inositol is present in an amount of about 2 mg to about 244 mg per serving of the mood support extract.

6. The mood support composition of claim 1, further comprising:
an ashwagandha extract;
a folate; and
vitamin B12, wherein the vitamin B12 comprises methylcobalamin.

7. The mood support composition of claim 6, further comprising a saffron extract.

8. The mood support composition of claim 7, wherein the saffron extract is present in an amount of about 14 mg to about 56 mg per serving of the mood support composition.

9. The mood support composition of claim 6, wherein the ashwagandha extract is present in an amount of about 125 mg to about 1800 mg per serving of the mood support composition;
vitamin B12 is present in an amount of about 0.5 μg to about 100 μg per serving of the mood support composition; and
the folate is present in an amount of about 0.1 mg to about 2 mg DFE per serving of the mood support composition.

10. The mood support composition of claim 1, further comprising:
melatonin;
L-theanine;
GABA (gamma-aminobutyric acid); and
an ashwagandha extract.

11. The mood support composition of claim 10, wherein the melatonin is present in an amount of about 1 mg to about 10 mg per serving;
the L-theanine is present in an amount of about 25 mg to about 500 mg per serving;
the GABA is present in an amount of about 100 mg to about 1000 mg per serving; and
the ashwagandha extract is present in an amount of about 50 mg to about 1800 mg per serving.

12. The mood support composition of claim 10, wherein about 20% to about 60% w/w of the melatonin is released within a first period of time.

13. The mood support composition of claim 10, wherein about 40% to about 80% w/w of the melatonin is released over a second period of time following the first period of time.

14. The mood support composition of claim 1, wherein the lavender and lemon balm are present at a ratio of about 25:1 to about 1:1 lavender:lemon balm.

15. The mood support composition of claim 1, wherein the chamomile and inositol are present at a ratio of about 25:1 to about 1:25 chamomile:inositol.

16. The mood support composition of claim 1, wherein the mood support composition is formulated as a tablet, a capsule, or a soft gel.

17. A method of improving one or more aspects related to mood in a mammal, the method comprising administering to the mammal the mood support composition of claim 1.

18. The method of claim 17, wherein improving an aspect related to mood comprises one or more of improving emotional health, improving the ability to cope with stress, improving the activity of a brain messenger, improving emotional balance, improving general well-being, improving the restfulness of sleep, and improving physical and mental energy.

19. A method of reducing feelings of sadness and/or feelings of stress in a mammal, the method comprising administering to the mammal the mood support composition of claim 1.

20. A method of inhibiting GSK3α and/or GSK3β in a mammal, the method comprising administering to the mammal the mood support composition of claim 1.
